(19) United States
(12) Patent Application Publication
(10) Pub. No.: US 2011/0294698 A1
(43) Pub. Date: Dec. 1, 2011

(54) PROCESS FOR IDENTIFYING DRUGS FOR TREATING GASTROESOPHAGEAL REFLUX

(75) Inventors: Michael R. Ruggieri, King of Prussia, PA (US); Larry S. Miller, Bala Cynwyd, PA (US); Alan S. Braverman, Exton, PA (US); Anil K. Vegesna, Philadelphia, PA (US); Mary F. Barbe, Dresher, PA (US)

(73) Assignee: Temple University of the Commonwealth System of Higher Education, Philadelphia, PA (US)

(21) Appl. No.: 13/165,133

(22) Filed: Jun. 21, 2011

Related U.S. Application Data
(63) Continuation-in-part of application No. PCT/US2009/069203, filed on Dec. 22, 2009.
(60) Provisional application No. 61/140,243, filed on Dec. 23, 2008.

(51) Int. Cl. C40B 30/06; G01N 33/566; G01N 33/74; C12Q 1/02

(52) U.S. Cl. 506/10; 435/29; 435/7.21

(57) ABSTRACT

Methods for identifying modulators of gastroesophageal smooth muscle relaxation include isolating various types of smooth muscle fibers from the stomach or esophagus and inducing the fibers to contract. The isolated and contracted fibers are used to screen test compounds for the compound's capacity to modulate relaxation of the smooth muscle fibers. In addition, newly identified unique nicotinic acetylcholine receptors are expressed in a cell, and used to screen test compounds for the compound's capacity to modulate the biological activity of the receptors.

### CROSS REFERENCE TO RELATED APPLICATIONS

This application is a continuation-in-part under 35 U.S.C. §§120 and 365(c) of International Application PCT/US2009/069203, filed on Dec. 22, 2009, and published as WO 2010/075388 on Jul. 1, 2010. This application also claims priority under 35 U.S.C. §119 to U.S. Provisional Application No. 61/140,243, filed Dec. 23, 2008. The entire contents of PCT/US2009/069203 and 61/140,243 are hereby incorporated by reference in their entirety for all purposes.

### STATEMENT OF FEDERALLY SPONSORED RESEARCH

Research leading to the disclosed inventions was funded, in part, with funds from the National Institutes of Health, grant no. R01 DK059500. Accordingly, the United States government may have certain rights in the inventions described herein.

### FIELD OF THE INVENTION

The invention relates generally to the fields of cell biology and drug discovery. More specifically, the invention relates to identifying modulators of the gastric and/or esophageal smooth muscle cell receptors that can reverse or prevent the relaxation of these muscle cells that can play a role in gastroesophageal reflux disease.

### BACKGROUND OF THE INVENTION

Various publications, including patents, published applications, technical articles and scholarly articles are cited throughout the specification. Each of these cited publications is incorporated by reference herein, in its entirety.

Gastroesophageal reflux disease (GERD) is a highly prevalent disorder affecting up to 40% of adults in the United States. The effects of GERD include not only the uncomfortable symptoms of indigestion, heartburn, regurgitation and dysphagia but the much more serious and eventually life threatening sequelae including esophageal erosion, ulceration, stricture, Barrett's esophagus and adenocarcinoma. In addition, gastro esophageal reflux in infants is considered a potential major cause of sudden infant death syndrome. Evidence is mounting that the prevalence of GERD is increasing coincident with the increased incidence of obesity (a risk factor for GERD) and substantial increase in the incidence of esophageal adenocarcinoma over the last 30 years.

It is estimated that 50% of the emergency room visits in the United States for chest pain are of esophageal origin, the majority of which are due to GERD. The condition can cause extra esophageal manifestations such as laryngitis, chronic bronchitis, chronic cough, asthma and dental erosions.

Treatment of GERD is a large economic burden with nearly $2 billion annual spending on over the counter antacids and H₂ histamine receptor blockers. Another $10 billion annually is spent on prescription acid suppression medications such as proton pump inhibitors. Recent evidence, however, suggests that long term stomach acid suppression is associated with decreased calcium absorption, and can lead to increased risk of bone fractures.

Despite the fact that GERD symptoms are well served by the currently available pharmacologic and surgical treatments, pharmacologic treatments that reduce stomach acid do not prevent reflux. Although effective symptomatic treatments for GERD have been developed, there is a continuing need to develop treatment strategies to prevent gastroesophageal reflux.

### SUMMARY OF THE INVENTION

The invention features methods for identifying modulators of gastroesophageal smooth muscle relaxation. In one aspect, the methods comprise inducing a clasp fiber or sling fiber isolated from the gastric smooth muscle of a mammal to contract, contacting the fiber with an agent that induces the fiber to relax and a test compound, and determining a modulation of the relaxation of the fiber in the presence of the test compound relative to the relaxation of the fiber in the absence of the test compound. In one aspect, the methods comprise inducing a lower esophageal circular fiber, a mid esophageal circular fiber, or a mid esophageal longitudinal fiber isolated from the esophageal smooth muscle of a mammal to contract, contacting the fiber with an agent that induces the fiber to relax and a test compound, and determining a modulation of the relaxation of the fiber in the presence of the test compound relative to the relaxation of the fiber in the absence of the test compound.

The agent that induces the fiber to relax is preferably a nicotinic acetylcholine receptor agonist, which can include hormones, biomolecules, or chemical compounds. In some detailed aspects, the agonist is acetylcholine, carbachol, or nicotine. The fibers can be contracted, for example, by contacting the fiber with muscarinic receptor agonist such as bethanechol. The fiber can be isolated from any mammal, and is preferably isolated from a human cadaver.

The fiber can be contacted with the relaxation agent before or after contacting the fiber with a test compound, or can be contacted with the relaxation agent and the test compound substantially at the same time.

The methods can employ the use of negative and positive controls. Thus, for example, in some aspects, the methods further comprise contacting the fiber with an agent that induces the fiber to relax and a negative control compound to provide a reference value for the level of modulation of the relaxation of the fiber induced by the test compound, or further comprise contacting the fiber with an agent that induces the fiber to relax and a positive control compound to provide a reference value for the level of modulation of the relaxation of the fiber induced by the test compound.

Also featured are methods for identifying modulators of gastroesophageal smooth muscle relaxation which comprise expressing at least one nicotinic acetylcholine receptor subunit in a cell, contacting the receptor with a test compound, and determining a modulation of the biological activity of the receptor in the presence of the test compound relative to the biological activity of the receptor in the absence of the test compound. The nicotinic acetylcholine receptor can comprise one or more subunits, and can, for example, comprise at least three or four subunits. In preferred methods, the receptor comprises at least five subunits. Each subunit of the receptor can independently comprise an alpha subunit, a beta subunit, a gamma subunit, a delta subunit, or an epsilon subunit. In some aspects, the methods further comprise contacting the receptor with a nicotinic acetylcholine receptor agonist. The method of cell can be any type of cell, and is preferably a eukaryotic cell such as a yeast cell, a mammalian cell, or an insect cell. The methods are preferably adapted for high throughput screening.

The receptor can comprise at least one, but preferably comprises at least two alpha subunits, at least one beta subunit, at least one gamma subunit, and at least one delta subunit, or can comprise at least two alpha subunits, at least one beta subunit, at least one epsilon subunit, and at least one delta subunit. In some aspects, the alpha subunit can be an alpha-2 subunit, an alpha-3 subunit, an alpha-4 subunit, an alpha-5 subunit, an alpha-7 subunit, an alpha-9 subunit, or an alpha-10 subunit. In some aspects, the beta subunit can be a beta-2 subunit.

### BRIEF DESCRIPTION OF THE DRAWINGS

FIG. 1 shows a screen of the Kay-Elemetrics workstation showing the ultrasound image and the corresponding manometric pressure (46.0 mmHg). The ultrasound image on the left is shown at the time indicated by the vertical line on the manometry tracing (right). The arrows points to the crural diaphragm.

FIGS. 2A and 2B show the inspiratory and expiratory pressure curves in normal volunteers. There are two pressure peaks in the subtraction curves in both inspiration (FI) (FIG. 2A) and expiration (FE) (FIG. 2B). The upper or more proximal peak reflects an “upper LES” or proximal intrinsic muscarinic cholinergic component. The distal peak reflects a “lower LES” or distal intrinsic muscarinic cholinergic component. The area under the pressure curve from the atropine resistant crural diaphragm was measured from the beginning of the upslope to the point where the down slope of the pressure curve crossed the zero pressure baseline (area not shaded). The lower intrinsic muscarinic cholinergic smooth muscle (atropine attenuated) area under the pressure curve was measured from the beginning of the upslope of the subtraction curve to the first minimum. The upper intrinsic muscarinic cholinergic smooth muscle area under the pressure curve was measured from the beginning of the upslope of the pressure curve after the first minimum to the tubular esophagus above the high-pressure zone. The area under the graphs of the pressure curves of the GERD subjects were performed in a similar manner.

FIG. 3 shows the averaged pressure distributions from pull-throughs referenced to the lower margin of the crus muscles. FIG. 3A shows full inspiration in normal volunteers. FIG. 3B shows full expiration in normal volunteers. FIG. 3C shows full inspiration in GERD patients. FIG. 3D shows full expiration in GERD patients.

FIG. 4A shows the ensemble averaged pressure curve in full expiration for the GERD patients, referenced to RCd. FIG. 4B shows the ensemble averaged pressure curve in full expiration for the normal control subjects.

FIG. 5 shows the pre EndoCinch, pre minus post atropine subtraction curves for seven GERD patients undergoing simultaneous ultrasound and manometry during FI.

FIG. 6 shows the pre EndoCinch, pre minus post atropine subtraction curves for seven GERD patients undergoing simultaneous ultrasound and manometry during FE.

FIG. 7 shows the post Endocinch pre minus post atropine subtraction curve during FE.

FIG. 8 shows the post Endocinch pre minus post atropine subtraction curve during FI.

FIG. 9A shows a 3D reconstruction of an endoscopic plication. The endoscopic placation appears as a spherical hyopechoic (dark) structure within the brighter mixed echoic mucosa/submucosa complex. FIG. 9B shows a 2D image of an endoscopic plication. The area appears as a hypoechoic round structure. In this picture, a suture within the plication is imaged in two planes on two dimensional ultrasound. The bright line represents a longitudinal image of the suture while the bright dot represents a cross sectional image of the suture. FIG. 9C shows a 3D image of the plication appears as a spherical hypoechoic area containing two linear hyperechoic sutures.

FIG. 10 shows the area of three plications graphed pressure vs. length.

FIG. 11 shows a concentration response curve to the selective muscarinic receptor against bethanechol (left) or the muscarinic and nicotinic agonist carbachol (right).

FIGS. 12A, 12B, and 12C show the results of mecamylamine, hexamethonium, and L-NAME inhibition of carbachol-induced relaxation of LEC (FIG. 12A), sling (FIG. 12B), and clasp (FIG. 12C), human muscle strips. Because of inequality of variances, non-parametric statistics were used (Mann-Whitney U tests)*=p<0.05; **=p<0.01.

FIG. 13 shows results of potential inhibition of nicotine-induced relaxation of human sling fibers.

FIG. 14 shows carbachol induced contraction and relaxation in human clasp (FIG. 14A) and sling (FIG. 14B) fibers in GERD patients and normal subjects.

FIG. 15 shows carbachol induced contraction (FIG. 15A) and relaxation (FIG. 15B) in human clasp, sling fibers, and LEC fibers in definite GERD patients, normal subjects (non-GERD), and probable GERD patients.

FIG. 16 shows concentration response curves for bethanechol induced contraction of human clasp fibers in the presence of various concentrations of darifenacin (DAR). Inhibition of bethanechol induced human clasp fiber contractions with increasing concentrations of the M₃ selective antagonist darifenacin causes parallel dextral shifts in the concentration response curve. Results are shown as percent of the maximal response shown in table 4. Control, n=14 strips from 4 donors; 30 nM DAR, n=6 strips from 2 donors, 100 nM DAR, n=5 strips from 2 donors; and 300 nM DAR, n=7 strips from 2 donors.

FIG. 17 shows concentration response curves for bethanechol induced contraction of human clasp fibers in the presence of various concentrations of methoctramine (METH). Inhibition of bethanechol induced human clasp fiber contractions with increasing concentrations of the M₂ selective antagonist methoctramine causes parallel dextral shifts in the concentration response curve. Results are shown as percent of the maximal response shown in table 4. Control, n=14 strips from 4 donors; 1 μM METH, n=3 strips from 1 donor; and 10 μM METH, n=3 strips from 1 donor.

FIG. 18 shows bethanechol induced clasp fiber contraction as a function of M₂ and M₃ receptor occupancy. The human clasp fiber bethanechol concentration response curve was converted into occupation response curves for the M₂ and the M₃ receptor subtypes. The y axis is the percent of the maximal bethanechol effect, the lower x axis shows the density of M₂ receptor occupied by bethanechol, while the upper x axis shows the density of M₃ receptors occupied. Receptor occupation=[A][R]/([A]+K_{A}), where [R] denotes the receptor concentration, K_{A} is the agonist dissociation constant (reciprocal of affinity) and [A] is the agonist concentration. Published values of K_{A} were used for bethanechol as follows: K_{A} for M₂=170 μM and KA for M₃=110 μM.

FIG. 19 shows a thee-dimensional graph of bethanechol induced clasp fiber contraction as a function of M₂ and M₃ receptor occupancy.

FIG. 20 shows a surface plot of clasp fiber contraction as a function of M₂ and M₃ receptor occupancy. Subtype selective antagonists alter the number of M₂ and M₃ receptors occupied by bethanechol which yield a given effect level. Using the formula for occupancy of an agonist in the presence of an antagonist (receptor occupancy=AR/(A+Ka(1+B/Kb))) and published antagonist affinity values, the M₂ and M₃ occupancy-effect curves in the presence of 3 concentrations of darifenacin and 2 concentrations of methoctramine were derived. A surface plot showing the effect of combinations of M₂ and M₃ occupancy in human clasp fibers is overlaid. The surface plot was constructed by transformation of the individual data points into a matrix using a random gridding method with Kringing correlation (Origin, Origin Lab Corp., Northampton, Mass.).

FIG. 21 shows a surface plot of LEC fiber contraction as a function of M₂ and M₃ receptor occupancy.

FIG. 22 shows a three-dimensional plot for M₂ and M₃ occupation and contractile response in the human clasp and sling (FIG. 22A and FIG. 22C, respectively) and pig clasp and sling (FIG. 22B and FIG. 22D, respectively)

FIG. 23 shows representative tracing of experimental paradigm.

FIG. 24 shows a comparison of the effect of nicotinic receptor blockers to inhibit cholinergic mediated relaxation of human (FIG. 24A) and pig (FIG. 24B) clasp fibers.

FIG. 25 shows carbachol induced relaxation of human sling fibers.

FIG. 26 shows the results of real time polymerase chain reaction (PCR) for the alpha and beta nicotinic receptor subunits mRNA in human gastro esophageal tissue dissections. The cycle when the fluorescence increased significantly above background (threshold cycle) was determined in duplicate for each of the nicotinic receptor subunits (alpha 1, 2, 3, 4, 5, 6, 7, 9 and 10 and beta 2, 3, and 4); alpha actin was used as a positive control. A tissue was considered positive for a subunit if either of the duplicate determinations had a threshold cycle of less than 45.

FIG. 27 shows immunofluorescent localization of nicotinic receptor subunits on specific cell types in human clasp fibers using specific antibodies for each subunit.

FIG. 28 shows pharmacologic specificity of nicotinic receptor-mediated relaxation of muscarinic receptor pre-contracted human gastric clasp (FIG. 28A) and sling (FIG. 28B) fibers.

FIG. 29 shows 1 mM nicotine induced relaxation of 30 μM bethanechol pre-contracted clasp, sling, and LEC muscle fibers before (open bars) and after (shaded bars) exposure to nicotinic receptor antagonist or vehicle, and 10 mM choline induced relaxation of 30 μM bethanechol pre-contracted strips (hatched bars).

### DETAILED DESCRIPTION OF THE INVENTION

It is to be understood that this invention is not limited to particular methods, reagents, compounds, compositions, or biological systems, which can, of course, vary. It is also to be understood that the terminology used herein is for the purpose of describing particular aspects only, and is not intended to be limiting.

The following abbreviations are used throughout the specification. GEJHPZ: gastroesophageal junction high pressure zone; GERD: gastroesophageal reflux disease; LEC: lower esophageal circular; LES: lower esophageal sphincter; MEC: mid esophageal circular; MEL: mid esophageal longitudinal; NAcR: Nicotinic Acetylcholine Receptor; TLESR: transient lower esophageal sphincter relaxations.

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value.

“Gastric” refers to the stomach and any subsection thereof.

“Gastroesophageal” refers to the stomach and esophagus and any subsection thereof.

A cell has been “transformed” or “transfected” by exogenous or heterologous nucleic acids such as DNA when such DNA has been introduced inside the cell. The transforming DNA may or may not be integrated (covalently linked) into the genome of the cell. In prokaryotes, yeast, and mammalian cells, for example, the transforming DNA may be maintained on an episomal element such as a plasmid. With respect to eukaryotic cells, a stably transformed cell, or “stable cell” is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

As used herein, “test compound” refers to any purified molecule, substantially purified molecule, molecules that are one or more components of a mixture of compounds, or a mixture of a compound with any other material that can be analyzed using the methods of the present invention. Test compounds can be organic or inorganic chemicals, or biomolecules, and all fragments, analogs, homologs, conjugates, and derivatives thereof. Biomolecules include proteins, polypeptides, nucleic acids, lipids, monosaccharides, polysaccharides, and all fragments, analogs, homologs, conjugates, and derivatives thereof. Test compounds can be of natural or synthetic origin, and can be isolated or purified from their naturally occurring sources, or can be synthesized de novo. Test compounds can be defined in terms of structure or composition, or can be undefined. The compound can be an isolated product of unknown structure, a mixture of several known products, or an undefined composition comprising one or more compounds. Examples of undefined compositions include cell and tissue extracts, growth medium in which prokaryotic, eukaryotic, and archaebacterial cells have been cultured, fermentation broths, protein expression libraries, and the like.

The term “express,” “expressed,” or “expression” of a nucleic acid molecule refers to the biosynthesis of a gene product.

As used herein, the terms “modulate” means any change, increase, or decrease in the amount, quality, or effect of a particular biological activity. “Modulators” refer to any inhibitory or activating molecules identified using in vitro and in vivo assays, e.g., agonists, antagonists, and their homologs, including fragments, variants, and mimetics, as defined herein, that exert substantially the same biological activity as the molecule. “Inhibitors” or “antagonists” are modulating compounds that reduce, decrease, block, prevent, delay activation, inactivate, desensitize, downregulate the biological activity or expression of a molecule or pathway of interest, or otherwise slow a biological response. “Inducers,” “activators” or “agonists” are modulating compounds that increase, induce, stimulate, open, activate, facilitate, enhance activation, sensitize, upregulate a molecule or pathway of interest, or otherwise facilitate a biological response. In some preferred aspects of the invention, the level of inhibition or upregulation of the expression or biological activity of a molecule or pathway of interest refers to a decrease (inhibition or downregulation) or increase (upregulation) of greater than from about 50% to about 99%, in some aspects, from about 60% to about 85%, in some aspects, from about 65% to about 85%, in some aspects, from about 70% to about 90%, in some aspects, from about 80% to about 95%, and in some aspects, from about 85% to about 99%. The inhibition or upregulation may be direct, i.e., operate on the molecule or pathway of interest itself, or indirect, i.e., operate on a molecule or pathway that affects the molecule or pathway of interest.

It has been discovered in accordance with the present invention that the gastroesophageal junction high pressure zone in GERD patients differs dramatically from the high pressure zone in healthy subjects. It has further been discovered that unique nicotinic acetylcholine receptors (NAcR) are present in the stomach and esophagus smooth muscle proximal to gastroesophageal junction. Without intending to be limited to any particular theory or mechanism of action, it is believed that the makeup of these NAcR may be a factor in susceptibility to the pressure zone differential, and may thus play a role in susceptibility to GERD. It is thus an object of the present invention to identify compounds that modulate the biological activity of the smooth muscle cells and fibers at or near the gastroesophageal junction such that inopportune relaxation of these muscles can be avoided or reversed. More particularly, it is an object of the present invention to identify compounds that can treat or prevent GERD. Accordingly, the invention features methods to identify compounds that modulate relaxation of the smooth muscle tissue in the stomach and/or esophagus.

In one aspect, the methods comprise isolating a clasp fiber from the gastric smooth muscle of an animal, inducing the clasp fiber to contract, contacting the clasp fiber with an agent that induces the clasp fiber to relax and a test compound, and determining a modulation of the relaxation of the clasp fiber in the presence of the test compound relative to the relaxation of the clasp fiber in the absence of the test compound. In another aspect, the methods comprise isolating a sling fiber from the gastric smooth muscle of an animal, inducing the sling fiber to contract, contacting the sling fiber with an agent that induces the sling fiber to relax and a test compound, and determining a modulation of the relaxation of the sling fiber in the presence of the test compound relative to the relaxation of the sling fiber in the absence of the test compound.

In another aspect, the methods comprise isolating a lower esophageal circular fiber from the esophageal smooth muscle of an animal, inducing the lower esophageal circular fiber to contract, contacting the lower esophageal circular fiber with an agent that induces the lower esophageal circular fiber to relax and a test compound, and determining a modulation of the relaxation of the lower esophageal circular fiber in the presence of the test compound relative to the relaxation of the lower esophageal circular fiber in the absence of the test compound. In another aspect, the methods comprise isolating a mid esophageal circular fiber from the esophageal smooth muscle of an animal, inducing the mid esophageal circular fiber to contract, contacting the mid esophageal circular fiber with an agent that induces the mid esophageal circular fiber to relax and a test compound, and determining a modulation of the relaxation of the mid esophageal circular fiber in the presence of the test compound relative to the relaxation of the mid esophageal circular fiber in the absence of the test compound. In another aspect, the methods comprise isolating a mid esophageal longitudinal fiber from the esophageal smooth muscle of an animal, inducing the mid esophageal longitudinal fiber to contract, contacting the mid esophageal longitudinal fiber with an agent that induces the mid esophageal longitudinal fiber to relax and a test compound, and determining a modulation of the relaxation of the mid esophageal longitudinal fiber in the presence of the test compound relative to the relaxation of the mid esophageal longitudinal fiber in the absence of the test compound.

The clasp and/or sling fibers can be isolated from the stomach according to any means suitable in the art. The lower esophageal circular fibers, mid esophageal circular fibers, and/or mid esophageal longitudinal fibers can be isolated from the esophagus according to any means suitable in the art. For example, the fibers can be isolated by dissection or microdissection. The fibers can be isolated from the stomach or esophagus of any animal, with mammals being preferred. Non-limiting examples of mammals include pigs, horses, cows, dogs, cats, rabbits, rats and mice. Particularly preferred are human cadavers. Once isolated, the fibers can be maintained in any suitable orientation, media, and/or environmental condition.

The fibers can be induced to contract by any suitable means, including electrical- or chemical-induced contraction. It is preferable that the contraction be maintained for a period of time sufficient to use the fibers in the screening assays. Any inorganic, organic, or biomolecule agent now known or later discovered to be capable of contracting smooth muscle fibers can be used. Non-limiting examples of suitable chemical agents include muscarinic receptor agonists. Other contractile agents include angiotensin II type 1 receptor agonists, histamine receptor agonists, neurokinin receptor agonists, alpha adrenergic receptor agonists or purinergic receptor agonists. Non-limiting examples of suitable muscarinic receptor agonists include bethanechol, muscarine, pilocarpine, McN-A-343, oxotremorine, carbachol, and acetylcholine. Non-limiting examples of suitable agonists of other receptors include 2-pyridylethylamine, 2-thiazolyethylamine, clobenpropit and imetit for histamine receoptors, beta-Ala(8)-neurokinin (NK) A (4-10), neurokinin B, and substance P for neurokinin receptors, phenylephrine, synephrine, and B-HT 920 for alpha adrenoceptors, and adenosine triphosphate (ATP), alpha, beta methylene ATP and beta, gamma methylene ATP for purinergic receptors.

The contracted fibers can then be contacted with at least one agent that induces relaxation of the contracted fibers and a test compound. In parallel, contracted fibers can be contacted with at least one agent that induces relaxation of the contracted fibers and an inert agent that is known not to antagonize the agent that induces relaxation in order to serve as a negative control, and other contracted fibers can be contacted with at least one agent that induces relaxation of the contracted fibers and an agent that is known to antagonize the relaxation agent in order to serve as a positive control or as a reference value. Contacting the fibers with both the relaxation agent and test compound is intended to identify test compounds that antagonize the relaxation agent, the relaxation agent's receptor, their interaction, or the biological response induced by their interaction, among other things. Thus, the test compound can interact with the relaxation agent to prevent binding to its cognate receptor, or can interact with the relaxation agent's receptor to prevent the relaxation agent from binding, or can inhibit or dampen the downstream biological activity, pathway, or response that is induced by the interaction of the relaxation agent the receptor.

The test compound can be contacted with the contracted fibers in advance of contacting the fibers with the relaxation agent, simultaneously with the relaxation agent, or after contacting the fibers with the relaxation agent. Various concentrations of the relaxation agent and/or test compound can be used.

Determining if the test compound modulates relaxation of the fibers can be determined according to any means suitable in the art. Preferably, the measurements are quantitative, and are based on a comparison of the level of relaxation induced in the presence or absence of the test compound. For example, smooth muscle strips can be pre-contracted with a muscarinic receptor agonist such as bethanechol, then induced to relax with a nicotinic agonist such as nicotine, cytisine, or epibatidine. Test compounds can be administered before inducing contraction to determine their effect on nicotine receptor activation induced relaxation as compared to strips tested without the test compound. Indirect indicators of relaxation can also be used, which would include monitoring the intracellular ion concentrations or ionic flux across cellular membranes by electrophysiologic recording techniques, radioactive ion flux such as 86Rb+, or by using ion sensitive dyes. Any measurable inhibition of the fiber's relaxation in the presence of the test compound can indicate that the test compound is an antagonist of relaxation. Any measurable enhancement of the fiber's relaxation can indicate that the test compound is an agonist of relaxation.

In one aspect, the methods comprise expressing at least one gastroesophageal nicotinic acetylcholine receptor in a cell. It is preferred that the receptor comprises at least two subunits, more preferably three subunits, more preferably four subunits, and most preferably five subunits that independently are an alpha subunit, beta subunit, gamma subunit, delta subunit, or epsilon subunit. Once expressed, the receptor can be contacted with a test compound, and a modulation of the biological activity of the receptor in the presence of the test compound relative to the biological activity of the receptor in the absence of the test compound can then be determined. In some aspects, the methods further comprise contacting the cell with a known nicotinic acetylcholine receptor agonist, before, concomitantly with, or after contacting the cell with the test compound. In this manner, determinations can be made as to whether the test compound further agonizes or antagonizes the known agonist.

Where the biological activity of the receptor treated with the test compound is higher than the activity in a receptor not treated with the test compound, the test compound is an agonist. Where the biological activity of the receptor treated with the test compound is lower than the activity in a receptor not treated with the test compound, the compound is an inverse agonist. Where the biological activity of the receptor treated with the test compound is not affected by an agonist the compound is an antagonist.

Gastroesophageal nicotinic acetylcholine receptors (NAcR) are heteromultimeric or homomultimeric complexes typically comprised of at least five subunits. Various subunits have been identified, and include, without limitation, the alpha subunit, the beta subunit, the gamma subunit, the delta subunit, and the epsilon subunit. NAcR subunits for expression in a cell can be derived from any animal, with mammalian NAcR subunits being preferred, and human NAcR subunits being particularly preferred. In humans, at least nine types of NAcR alpha subunits have been identified, and include alpha 2-7, 9, and 10, and at least three types of NAcR beta subunits have been identified, and include beta 2-4. The stoichiometry of these receptors in nuromuscular nictonic receptors is typically alpha₍₂₎/beta/gamma/delta, and the epsilon subunit replaces the embryonic gamma subunit in adulthood.

In some aspects, the gastroesophageal NAcR is comprised of only one alpha subunit. In some aspects, the gastroesophageal NAcR is comprised of at least two alpha subunits, at least one beta subunit, at least one gamma subunit, and at least one delta subunit. In other aspects, the gastroesophageal NAcR is comprised of at least two alpha subunits, at least one beta subunit, at least one epsilon subunit, and at least one delta subunit. The alpha subunits can be any of the alpha 1, 2, 3, 4, 5, 6, 7, 9, or 10 subunits. Particularly preferred alpha subunits can be selected from alpha 2, 3, 4, 5, 7, 9, and 10 subunits. The beta subunits can be any of the beta 2, 3, or 4 subunits. The beta-2 subunit is particularly preferred.

As discussed in Examples 13 and 14 below, it is believed that the gastroesophageal NAcR which mediates nicotine induced relaxation in clasp and LEC fibers has a pharmacologic profile consistent with either a type II NAcR (i.e., receptors that include alpha-4 beta-2 subunits) or a type IV NAcR (i.e., receptors that include alpha-2 beta-4/alpha-4 beta-4 subunits), and that the gastroesophageal NAcR which mediates nicotine induced relaxation in sling fibers has a pharmacologic profile consistent with either a type III NAcR (i.e., receptors that include alpha-3 beta-4 beta-2 subunits) or a type IV NAcR (i.e., receptors that include alpha-2 beta-4/alpha-4 beta-4 subunits). Alternatively, the actual subtype of nicotinic receptor mediating relaxation in clasp, sling, and LEC fibers may be unique and may not be either type II, type III, or type IV. A particularly preferred gastroesophageal NAcR is a type II, type III, or type IV gastroesophageal NAcR, or a gastroesophageal NAcR that has a pharmacologic profile consistent with a type II, type III, or type IV gastroesophageal NAcR.

In one aspect, the methods according to the present invention comprise expressing at least one gastroesophageal NAcR in a cell, wherein the at least one gastroesophageal NAcR is a type II, type III, or type IV gastroesophageal NAcR, or is a gastroesophageal NAcR that has a pharmacologic profile consistent with a type II, type III, or type IV gastroesophageal NAcR. Once expressed, the receptor can be contacted with a test compound, and a modulation of the biological activity of the receptor in the presence of the test compound relative to the biological activity of the receptor in the absence of the test compound can then be determined. In some aspects, the methods further comprise contacting the cell with a known nicotinic acetylcholine receptor agonist, before, concomitantly with, or after contacting the cell with the test compound. In this manner, determinations can be made as to whether the test compound further agonizes or antagonizes the known agonist. The gastroesophageal NAcR can be expressed in any cell suitable in the art, including cells isolated from relevant gastroesophageal tissue and established cell lines. The cells can be produced by transfecting the cell with the relevant gastroesophageal NAcR genes. Preferably, the cell is a stable cell. Prokaryotic or eukaryotic cells can be used, with eukaryotic cells being preferred. Non-limiting examples of such cells include yeast, insect, and mammalian cells. Specific types of such cells may include any suitable cell types such as Xenopus (frog) oocytes, Chinese Hamster Ovary (CHO) cells, human embryonic kidney (HEK) cells, human epithelial cells, human neuroblastoma cells or any such cell type capable of being transfected. Immortalized cell lines are preferred. Cells can be transiently transfected with the nicotinic receptor subunits (either singly or in combination) or stably transfected to create cell lines stably transfected with different combinations of nicotinic receptor subunits.

The test compound can be contacted with the cell according to any means suitable in the art. The effect of the test compound on the biological activity of the NAcR an be determined by any means suitable in the art. The test compound can be assessed at multiple concentrations. In some aspects, the test compound is assessed for its ability to modulate at least one biological activity of the gastroesophageal NAcR. In preferred aspects, the biological activity is to modulate relaxation of the smooth muscle tissue of the stomach and/or esophagus.

For example, the biological activity of the gastroesophageal NAcR can be determined by measuring the current across the cell induced by activation of the NAcR in voltage clamped cell preparations. Voltage clamping is one preferable technique to measure NAcR current. Voltage clamp techniques are well known in the art. The following parameters can be measured using a voltage clamp: single channel conductance, channel open time, voltage dependence, blockade induced by application of a particular compound, and activation induced by application of a particular compound. Other suitable techniques for measuring the biological activity of gastroesophageal NAcR include radiolabeled ion flux assays, patch clamping, voltage-sensitive dyes, and ion-sensitive dyes. For example, changes in intracellular sodium ions induced by nicotinic receptor activation can be monitored using dyes available from commercial suppliers such as Molecular Probes®, (Ivitrogen, Carlsbad, Calif.) including such dyes as SBFI, Sodium Green Na⁺ Indicator, CoroNa™ Green Na+ Indicator, CoroNa™ Red Na⁺ Indicator, and the like. The nicotinic receptor mediated ion channel flux can also be monitored using fluorescence detection techniques in a microtiter plate format or by flow cytometry. All such assays are well known in the art.

The invention also features high-throughput screening assays to identify compounds that modulate the biological activity of gastroesophageal NAcR. High-throughput screening assays are useful for screening of large numbers of test compounds in an efficient manner. For example, but not by way of limitation, cells expressing a gastroesophageal NAcR can be seeded throughout a multi-well plate such as a 96-well microtiter plate. Each well of the microtiter plate can be used to run a separate assay against a candidate compound. A microtiter plate permits screening of multiple concentrations of a test compound, and multiple test compounds, alone or in combination with other test compounds. The assays may take place in the presence of additional agonists or antagonists. Data obtained for the test compounds are compared with measurements taken in the presence of known agonists or antagonists and/or to control samples (such as a non-stimulatory/non-inhibitory medium).

The following examples are provided to describe the invention in greater detail. They are intended to illustrate, not to limit, the invention.

### Example 1

### Induced Transient Lower Esophageal Sphincter Relaxation Occurs at a Lower Gastric Pressure and Volume in GERD Patients than in Normal Control Subjects

These studies were carried out to develop a novel method to trigger transient lower esophageal sphincter relaxations (TLESRs) using gastric distention, and determine the intragastric pressure threshold for inducing TLESR in normal control subjects with and without a normal gastroesophageal junction.

### Material and Methods.

Subjects. This was a nonrandomized, controlled trial with blinded ascertainment of outcomes. The study consisted of a total of eighteen volunteers (13 men and 5 women; mean age of 33±7.5 years). Temple University Institutional Review Board approved the study protocol, and informed consent was obtained from all participants. None of the subjects had a history of surgical manipulation of the upper GI tract. The subjects did not have any abdominal symptoms.

Exclusion criteria included subjects on any medication that could affect the gastroesophageal segment high-pressure zone, including the use of antacids, H₂ blockers, proton pump inhibitors, prokinetic agents, erythromycin type antibiotics and anticholinergics. The following were also exclusion criteria: GI symptoms, GERD, hiatal hernia, conditions and disorders including a history of esophagitis, gastrointestinal symptoms such as abdominal pain, heartburn, regurgitation, chest pain, difficulty swallowing, pain on swallowing, dysphagia, abdominal surgery involving the stomach or esophagus, nausea or vomiting, diabetes, scleroderma, esophageal motility disorders, non-cardiac chest pain, achalasia and current pregnancy. There were no dropouts in the study as this was a single visit outpatient study.

Endoscopic evaluation of the study subjects. All subjects underwent upper endoscopy after an overnight fast with an Olympus GIF H180 endoscope or Pentax EG endoscope. Subjects were kept in the left lateral decubitus position during the procedure. Cetacaine spray was used to anesthetize the posterior pharyngeal wall as the endoscopy was performed unsedated. Subjects were evaluated for the presence of esophagitis and for any abnormalities in stomach and duodenum including hiatal hernia. The entire procedure was videotaped for all the subjects.

After viewing the esophagus, stomach and duodenum, a water perfused manometric catheter was passed through the biopsy channel of the endoscope. This manometry catheter was continuously perfused with gas-free distilled water by a low compliance pneumohydraulic capillary infusion system (Arndorfer Medical Specialties, Greendale, Wis.) at a rate of 0.5 ml/min. Air was insufflated through the biopsy channel of the endoscope at a constant air flow of 24 ml per second without any pulses during the procedure.

Manometric Evaluation. Manometric studies were performed using a single port water perfusion manometric catheter, which was passed through the biopsy channel of the endoscope. After visualizing the stomach all the air in the stomach was removed by suction through the endoscope. Any remaining air from the cardia of the stomach was removed and a baseline pressure was recorded keeping the endoscope in a retroflexed position along the lesser curvature side of the stomach. After having a steady baseline pressure for few minutes air was insufflated into the stomach continuously at a constant flow through the endoscope until the gastroesophageal junction (GEJ) opened or until the subjects complained of discomfort. The stomach was kept deflated to measure a steady stomach baseline pressure for 15 to 30 seconds between each gastric distention sequence. This was repeated 5-6 times in order to exclude trials in which the pressure readings were abnormal due to abdominal contraction, belching or due to swallows.

A dental suction device was placed in the subject's mouth to prevent accumulation and swallowing of saliva. In addition an acoustic monitor was placed on the subject's neck to assess for swallowing and burping. The whole study was recorded on a Kay Elemetrics swallowing workstation. This system was used to synchronize the pressure readings to the video recording of the endoscopic procedure. During the study the subject was monitored to see if there were any swallows. As this was an unsedated study the subject was also asked to notify the team if he/she swallowed during the study. If swallows were observed, those trials were discarded and were not included in the analysis.

The studies were analyzed from recorded video images and pressure recordings on the Kay Elemetrics swallowing workstation in a blinded manner. The video images were evaluated to determine opening of the hiatus without knowledge of the pressure at that time point. Average pressure was then obtained at that time point for each individual subject for the five to six gastric distentions that were performed.

Statistical Analysis. Results are presented as means±S.E. The variables were compared between groups using a T-test. For all the studies, an associated probability (p value) of less than 0.05 was considered statistically significant. The power of the study for this sample size is 80%.

### Results.

Out of 18 normal subjects, 5 were found to have hiatal hernia during the endoscopy. The 13 normal volunteers without hiatal hernias did not have any abnormalities of the esophagus, stomach or duodenum. Three patients with GERD without hiatal hernia were also evaluated (3 males, average age 42 years old).

In the 13 normal volunteers without hiatal hernias the stomach was inflated for an average period of 26 seconds during each insufflation. Two patterns of gastro-esophageal junction opening in the thirteen normal volunteers without hernias were observed during the study. In pattern I (8 normal subjects) the hiatus slowly stretches and deforms; however the hiatus and distal esophagus opened simultaneously, allowing the expulsion of air from the stomach into the esophagus. The mean gastric pressure and volume at the point of distal esophageal opening in pattern I was 11.6±1.7 mmHg and 1284.15 mL+/−570.23 mL. After hiatal opening the gastric pressure dropped slightly by about 2-3 mm Hg. In pattern II (7 normal subjects, 2 of the normal subjects had overlap between pattern I and II during multiple distension studies) the hiatus opened rapidly after insufflation of air into the stomach. Then at some time point later and at a higher pressure, the distal esophagus opened allowing for expulsion of air into the esophagus. The mean gastric pressure and volume for opening in pattern II was 13.9±1.9 mmHg and 1228.59+/−763.97 mL for the hiatus and 19.7±1.9 mmHg and 1728.83 mL+/−660.34 mL for the distal esophagus. The mean length of time that the hiatus remained open on endoscopic visualization after the initiation of opening was 17.1±3.3 sec. for type I opening and 37.6±5.0 sec. for type II opening after the initial hiatal opening.

In the 5 normal volunteers with hiatal hernia, the gastroesophageal junction opened at a mean pressure and volume of 3.12 mmHg and 221 mL above the baseline, significantly lower than in the normal control subjects. (p<0.0001).

In the three GERD patients, the mean gastric pressure and volume at hiatal opening was 3 mmHg and 149.4±57.5 mL significantly lower than in the normal control subjects without hiatal hernia (p=0.021).

In all cases there was endoscopic evidence of esophageal body contractions after the distal body opened, in the absence of swallows, as viewed endoscopically. The time of hiatal opening recorded during swallows was usually less than 5 sec. In all cases there was endoscopic evidence of esophageal body contractions after the distal body opened in the absence of swallows.

### Example 2

### A Missing Sphincteric Component of the Gastro-Esophageal Junction in Patients with Gastroesophageal Reflux Disease

The purpose of these studies was to determine if there were any significant differences in the strength or relative positioning of the three sphincteric components in GERD patients.

Subjects. Fifteen normal volunteer subjects were studied (eight male, seven female, 23-47 years, mean age 34±8.5 years). Ten patients with GERD were evaluated. Eight of the ten GERD study patients responded to the Endocinch procedure with a reduction or elimination of their symptoms. Seven of these patients underwent pre and post Endocinch evaluation with simultaneous ultrasound and manometry before and one month after the Endocinch procedure. These seven patients (three male and four female) with GERD (33-66 years, mean age 45±10.7 years) were evaluated over the same time period with the same procedure as the normal subjects.

The GERD patients all complained of heartburn and/or regurgitation prior to the Endocinch procedure, which was relieved with high dose proton pump inhibitors. All subjects gave IRB approved informed consent to take part in the studies, and all subjects were tested in accordance with the policies of the National Institute of Health and Temple University School of Medicine. Exclusion criteria for all subjects included subjects on any medication which could affect the gastroesophageal junction high-pressure zone. This included prokinetic agents, erythromycin type antibiotics and anticholinergics. The following medical conditions were also considered exclusion criteria: abdominal surgery involving the stomach or esophagus, diabetes, scleroderma, achalasia and current pregnancy. In addition normal volunteers were excluded if they used antacids, H₂ blockers, proton pump inhibitors, had any Gastrointestinal symptoms, conditions and disorders including a history of esophagitis, abdominal pain, heartburn, reflux, regurgitation, chest pain, difficulty swallowing, pain on swallowing, dysphagia, nausea or vomiting, esophageal motility disorders or non cardiac chest pain.

Endoscopy. All study subjects underwent upper endoscopy using a Pentax 2900 video endoscope (Pentax, Orangeburg, N.Y., USA) using topical oral anesthesia with Cetacaine (Getylite Industries, Pennsauken, N.J., USA), with or without sedation. Subjects found to have a hiatal hernia were excluded from the normal group. Hiatal hernia was not an exclusion criterion in the GERD study group.

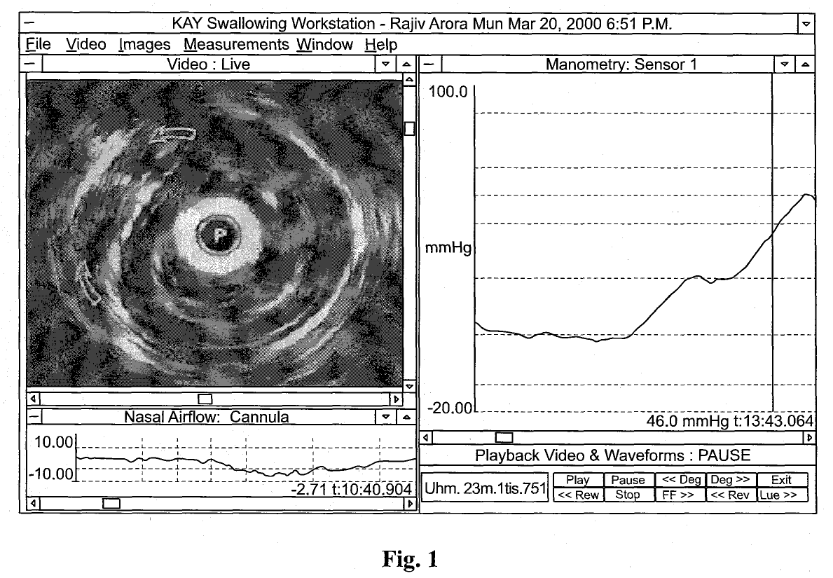

Equipment. A custom assembly was constructed which combined a 20 MHz ultrasound (US) transducer (Microvasive, Boston Scientific, Watertown Mass.) with a water perfused manometry catheter. The manometry catheter consisted of a 3 French angiography catheter with a small side hole port at the same level as the ultrasound transducer, to simultaneously obtain gastroesophageal junction high-pressure zone musculature cross-section images and corresponding intraluminal pressures at the same location. The transducer rotated at 15-30 Hz to provide 360 degree esophageal cross-section imaging with 0.1 mm axial slice thickness and a typical penetration of about 2 cm. Images were recorded on VHS videotape at 30 frames/sec on a Kay Elemetrics swallowing workstation (Kay-Elemetrics, N.J.) to provide temporal synchronization of the two data sources (FIG. 1).

A custom made pull-through machine provided a calibrated, constant retraction of the simultaneous ultrasound and manometry catheter in a proximal direction through the stomach and the esophagus at 0.5 cm/s. The pressure data were saved to a computer file and the US images digitized into 256 gray levels, 640×480 lossless TIFF files.

Procedure and data collection. Prior to insertion of the simultaneous ultrasound and manometry assembly into the proximal stomach, the back of each subject's throat was numbed with Cetacaine and the nose numbed with Lidocaine to reduce discomfort during the catheter's passage through the nostril. An intravenous line was prepared to allow for the later administration of atropine.

Ultrasound images were collected and co-localized with manometric pressure in the 15 healthy volunteer subjects and 7 GERD patients with breath holding under full inspiration (FI) and full expiration (FE) during a machine pull-through of the catheter assembly at 5 mm/s from the stomach into the thoracic esophagus. The subject lay supine with his or her back at approximately a 35-degree angle. Subject movement was minimized during the duration of the study. Ultrasound imaging verified that the initial transducer position was in the proximal stomach at both FI and FE. After ensuring the catheter's position was correct, the catheter was marked at the nares to ensure accurate repositioning of the transducer assembly in the stomach at the pull through start (PTS) reference location.

Sphincteric contributions to pressure were measured with the costal diaphragm in the extreme inferior and superior positions. For maximal inferior positioning, the subject was instructed to inhale as deeply as possible and hold his/her breath during ‘full-inspiration’ (FI) pull-throughs. For maximal superior positioning, the subject exhaled as far as possible and held his/her breath during ‘full-expiration’ (FE) pull-throughs. Each pull-through began in the stomach at the premarked start location of the transducer (pull-through start position) and ended well into the esophageal body. At least three pull-throughs were recorded for each FI and FE respiratory state. Swallowing was monitored, and if the subject swallowed at any time during the pull-through, the entire pull-through was discarded. Subjects were asked to hold their breath after deep inspiration or exhalation in order to quantify the changes in axial pressure variation associated with changes in alignment of smooth versus skeletal muscle sphincteric tone from inferior versus superior displacement of the costal diaphragm.

Pull-throughs were repeated after intravenous administration of atropine. For each pull-through the axial locations of the distal margin of the right crus muscle (RCd) were quantified to use as a spatial reference when averaging pressures over the 15 normal and 7 GERD patient subjects. In addition to the RCd, the initiation of the pull-through (PTS) was also used as a spatial reference to determine absolute displacement of the pressure peaks. For this reason great care was taken to return the catheter assembly to its original position by marking the position of the nares on the catheter with a Sharpie pen. The PTS reference was not used to evaluate shifts between the pre- to post-atropine pull-throughs, as the extended passage of time between data collection led to reference drift. All analysis was carried out with in-house computer software or image pro plus software (Image Pro plus version six, Media Cybernetics, Bethesda, Md.).

After collecting data in full inspiration and full expiration, the intrinsic muscarinic cholinergic smooth muscle contribution to the sphincter was attenuated (Fang J C et al. (1999) Gut, 44:603-607). These studies showed dose-dependent partial suppression of the resting sphincteric pressure by atropine. An initial bolus of atropine (15 ug/kg) was administered intravenously, followed by continuous intravenous atropine infusion at 4 ug/kg/h during the remainder of the study. After waiting 30 minutes and determining that there was an appropriate increase in heart rate to assure maximal suppression of cholinergic smooth-muscle tone (approximately 40% or greater over baseline heart rate), the data were collected for the same positions and respiratory states as done in the absence of atropine with three additional assembly pull-throughs.

Each subject had multiple insertions of the transducer assembly into the stomach and subsequent data collection during a constant speed retraction with the pull-through machine. Each of these insertions and retractions is defined as a “pull through.”

Data analysis. The crural sling could be clearly identified on the ultrasound images (FIG. 1). The crus muscles impinging on the esophageal wall appear as hypoechoic muscle bundles. The proximal (RCp) and distal (RCd) margins of the crural sling adjacent to the esophageal wall were identified, and checked independently, as the first and last extrinsic crus muscle bundles imaged during each pull-through. The ‘width’ of the crural sling was defined as the axial separation between RCd and RCp. To quantify relative anatomic shifts in crural sling location, both proximal and distal crus locations were used as references. There were no statistical differences in the results when using either reference. Therefore, all results use the distal marker (RCd) as the spatial reference for the crural sling. The spatial excursions of the anatomic crus and the pressure signatures between FI and FE were determined by referencing to the location of the transducer assembly in the stomach at the initiation of each pull-through (PTS). This was done both pre and post atropine. To quantify relative anatomical shifts in crural diaphragm location, the RCd and PTS were used.

From the three (or more) pull-throughs per subject for each case, one pull-through was chosen based on best quality of high frequency ultrasound images for determining the anatomic crural sling spatial references. After data collection, separate gastric baseline pressures were determined for each pull-through by averaging the pressure signal 5-10 s just prior to the start of each pull-through, with cough and other obvious artifacts excluded. All pressures were referenced to time averaged gastric baseline pressure.

Reconstructing the muscarinic cholinergic smooth-muscle (atropine attenuated) pressure distribution. As described above, the administration of atropine attenuates the muscarinic cholinergic components of smooth muscle tone in the gastro-esophageal high-pressure zone segment.

The intrinsic muscarinic cholinergic smooth muscle contribution to pressure was reconstructed by subtracting the post-atropine pressures from the full pre-atropine pressures after spatial referencing to the RCd. This subtraction process leaves the purely muscarinic cholinergic contribution to pressure since the pressure profile from the crural diaphragm and any residual intrinsic non-cholinergic pressure is removed in the subtraction process. In this way the full pressure distribution, the atropine-resistant and the atropine attenuated intrinsic cholinergic smooth muscle pressure distributions were obtained.

These pressures were averaged relative to the inferior margin of the anatomic crural sling when the costal diaphragm was in its extreme superior (FE) and inferior (FI) positions. The individual pressure profiles were linearly interpolated onto a grid with time increment of 1/250 s before ensemble averaging.

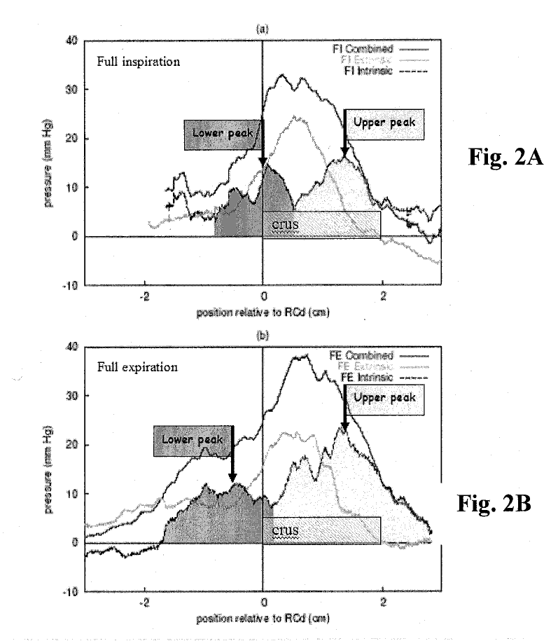

Measurement of area under the curve. In order to quantify the pressure contributions from each component of the gastroesophageal junction high-pressure zone, the area under the averaged pressure curve was measured for the GERD patients and the normal controls using image pro-plus software. The area under the crural diaphragm (atropine resistant) pressure curve was measured from the beginning of the upslope of the pressure curve to the point where the down slope of the pressure curve crossed the zero pressure baseline. The dower intrinsic muscarinic cholinergic smooth muscle (atropine attenuated) area under the pressure curve was measured from the beginning of the upslope of the subtraction curve to the first minimum. The upper intrinsic muscarinic cholinergic smooth muscle (atropine attenuated) area under the pressure curve was measured from the beginning of the upslope of the pressure curve after the first minimum to the tubular esophagus above the high-pressure zone (FIGS. 2A and 2B).

Statistics. All statistical tests were performed using the paired Student's T Test with 95% confidence level and assuming equal variances. Ensemble plots are presented from all 15 normal subjects and all 7 GERD patients. Data analysis included the percent contribution of the area under the curve (AUC) from intrinsic muscarinic cholinergic smooth muscle pressure profiles and the atropine resistant pressure profiles. The mean+/−SD of these values were reported. The above data were evaluated to determine the intrinsic muscarinic cholinergic smooth muscle (atropine attenuated) pressure profiles and the crural diaphragm (atropine resistant) contributions to the EGS and to determine the effects of respiration on the position and pressure relationships of these pressure profiles.

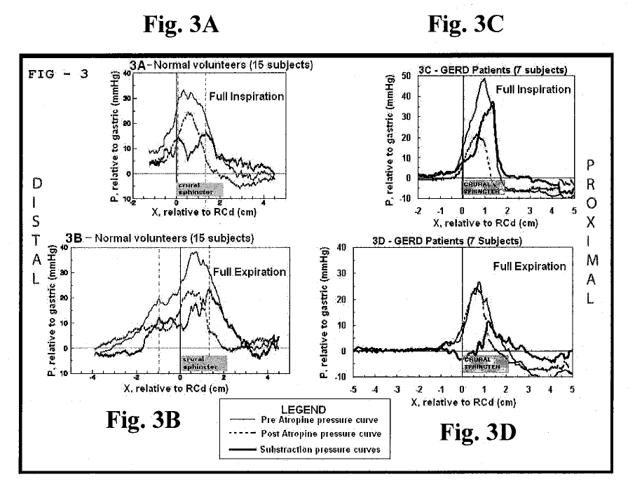

Analysis of normal control subjects. Fifteen normal volunteer subjects had normal esophageal and gastro-esophageal segment high-pressure zone function. The width of the longitudinal segment of the esophageal wall in contact with the crural sling averaged 2.0-2.3 cm, as measured from ultrasound imaging. The lower margin of the crural sling was displaced by 1.9 cm as the costal diaphragm shifted from its inferior-most (FI) to its superior-most (FE) respiratory positions. The ensemble averaged full pressure profiles of the high-pressure zone were compared with the averaged pressure profiles after administering atropine, in FI and FE. (FIGS. 3A and 3B). These plots lined up relative to the distal margin of the RCd.

In normal volunteer subjects the distal intrinsic muscarinic smooth muscle pressure component contributes 34% of the AUC of the entire pressure profile to the gastroesophageal junction high pressure zone in FI and 31% in FE (Table 1).

Table 1 demonstrates the percent contribution of each sphincteric component to the area under the pressure curve.

| | Full Inspiration | | Full Expiration | |
|---|---|---|---|---|
| | % of total | % of intrinsic | % of total | % of intrinsic |
| | area under the | area under the | area under the | area under the |
| | pressure curve | pressure curve | pressure curve | pressure curve |
| Upper LES | 25 | 66 | 43 | 69 |
| CD | 62 | NA | 39 | NA |
| Lower LES | 13 | 34 | 20 | 31 |

Analysis of GERD patients. Two of the GERD patients were found to have hiatal hernia at endoscopy (one small and one moderate sized hiatal hernia). The other five GERD patients showed no sign of hiatal hernia. The pre and post atropine pressure curves during full inspiration and full expiration in GERD patients are shown in FIGS. 3C and 3D. The pre minus the post atropine subtraction curves in the GERD patients demonstrated distinctly different pressure profiles from the subtraction curves in the normal volunteer subjects in both full inspiration and full expiration (FIGS. 3A and 3B). In the GERD patients the subtraction curve, demonstrated that the distal intrinsic muscarinic smooth muscle pressure peak (lower LES), which was present in the normal volunteer subjects (FIGS. 3A and 3B), was absent in both the inspiratory and expiratory phases in all the GERD patients, while the proximal intrinsic muscarinic smooth muscle pressure peak (upper LES) seen previously in the normal volunteer subjects was present and at the same axial position relative to the RCd as in the normal volunteer subjects.

In the GERD group, the width of the gastroesophageal junction high-pressure zone during FI was 2.5+/−0.7 cm and the width during FE was 2.5+/−0.7 cm (p=0.9). Unlike the normal volunteer subjects, there was no significant change in the width of the high-pressure zone between FI and FE. The width of the CD during FI was 2.1+/−0.8 and during FE was 2.1+/−0.6 (p=0.9). Like the normal control subjects, the width of the CD did not change between FI and FE. These results indicate that there was no lengthening of the gastroesophageal junction high-pressure zone from FI to FE in the GERD patients as opposed to the normal volunteer subjects. The beginning of the RCd moved 1.4 cm proximally relative to the initiation of the pull-through start position between FI and FE; the intrinsic muscarinic smooth muscle pressure profile moved approximately the same distance in concert with the CD. This is also approximately the same distance that the RCd moved between FI and FE in the normal volunteer subjects.

In the GERD patients the distal intrinsic muscarinic smooth muscle pressure component (lower LES) made no contribution to the pressure of the gastroesophageal junction high-pressure zone pressure profile (Table 2).

Table 2 demonstrates the percent contribution of each sphincteric component to the area under the pressure curve. Note that there is no distal intrinsic muscarinic pressure component in the GERD patients.

| | Full Inspiration | | Full Expiration | |
|---|---|---|---|---|
| | % of total | % of intrinsic | % of total | % of intrinsic |
| | area under the | area under the | area under the | area under the |
| | pressure curve | pressure curve | pressure curve | pressure curve |
| Upper LES | 62.9 | 100 | 26 | 100 |
| CD | 37.1 | NA | 74 | NA |
| Lower LES | 0 | 0 | 0 | 0 |

Discussion. The intrinsic sphincter (esophageal smooth muscle sphincter) and the crural diaphragm (external skeletal muscle sphincter) are anatomically superimposed in normal individuals. The intraluminal pressure is a summation of pressures from all of these muscle groups. Distinguishing the components of the distal esophageal high-pressure zone is important because these pressures reflect anatomic and/or physiologic components of the sphincter and because these pressures combine to equal the closure forces that contributes to and maintains the function of the anti-reflux barrier.

This example compared the gastroesophageal junction high-pressure zone pressure profile in GERD patients to the gastroesophageal junction high-pressure zone pressure profile in normal volunteer subjects. Using simultaneous ultrasound and manometry with and without atropine attenuation of the intrinsic muscarinic smooth muscle components of the high pressure zone three components of the gastroesophageal junction high-pressure zone were isolated in normal control subjects. Although the two intrinsic muscarinic pressure components may not represent 100% of the muscarinic tone generated (since atropine may not ablate all of the muscarinic tone), they are nevertheless generated by pure muscarinic tone since the smooth muscle contribution to the pressure was reconstructed by subtracting the post-atropine pressures from the pre-atropine pressures after spatial referencing to the RCd. This subtraction process leaves only a pure muscarinic contribution to the pressure since the pressure profile from the crural diaphragm and any residual intrinsic non-muscarinic pressure is removed in the subtraction process.

The three high-pressure zone components consist of a proximal intrinsic muscarinic smooth muscle pressure component (the upper LES), a distal intrinsic muscarinic smooth muscle component (the lower LES) and an atropine resistant pressure component (the external crural diaphragm). Each pressure component was localized spatially with respect to the other pressure components, and the percent contribution of each pressure component to the antireflux barrier gastroesophageal junction high-pressure zone was quantified by measuring the area under the ensemble averaged pressure curves. It was determined that the intrinsic proximal muscarinic smooth muscle component and the crural diaphragm move in lock step with each other during respiration and move away from the distal intrinsic muscarinic smooth muscle pressure component during full expiration, thus accounting for the lengthening of the high pressure zone between full inspiration and full expiration.

The high-pressure zone in GERD patients (FIGS. 3C and 3D) differs dramatically from the high pressure zone in normal control subjects (FIGS. 3A and 3B). The distal intrinsic muscarinic smooth muscle pressure profile that was demonstrated in the normal control subjects is absent in all GERD patients weather or not a hiatal hernia is present. In the normal volunteer subjects the gastroesophageal junction high-pressure zone pressure profile lengthens due to the proximal intrinsic muscarinic smooth muscle pressure component moving proximally away from the relatively fixed distal intrinsic muscarinic smooth muscle pressure component. While the results in the GERD patients also demonstrate respiratory movement of the intrinsic muscarinic smooth muscle pressure component and the crural diaphragm in lock step, the width of the gastroesophageal junction high-pressure zone pressure profile remained unchanged between FI and FE. The explanation for this is that there is no distal intrinsic muscarinic smooth muscle pressure component. Therefore when the intrinsic muscarinic smooth muscle pressure component and crural diaphragm move it does not lengthen the high-pressure zone (no distal component to move away from).

In normal volunteer subjects the distal intrinsic muscarinic smooth muscle pressure component contributes 34% of the AUC of the entire pressure profile to the gastroesophageal junction high pressure zone in FI and 31% in FE (Table 1). In the GERD patients the distal intrinsic muscarinic smooth muscle pressure component (lower LES) made no contribution to the pressure of the gastroesophageal junction high-pressure zone pressure profile (Table 2).

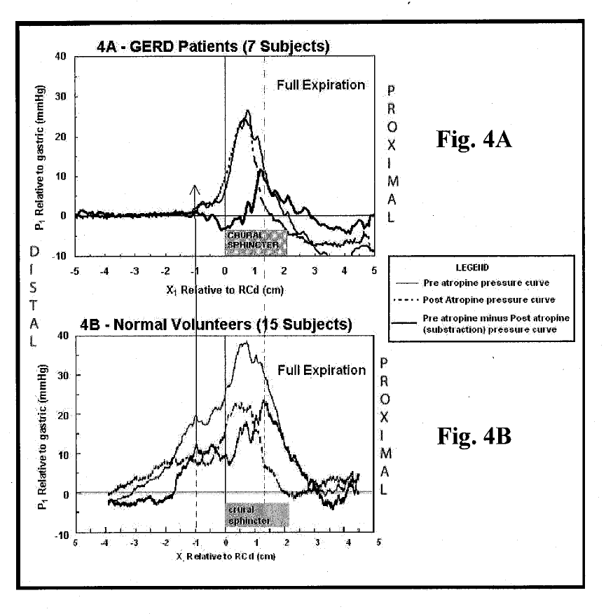

The proximal intrinsic muscarinic smooth muscle pressure profile appears to be a physiologic sphincter of esophageal circular smooth muscle. Given the close correspondence of the pressure contribution defining the upper LES in the normal group with the single pressure contribution in the GERD group, it is believed that these two pressure contributions arise from the same intrinsic muscarinic smooth muscle sphincteric component in both normal subjects and GERD patients. The belief is based on two observations. First, the location of this intrinsic muscarinic smooth muscle pressure component relative to the RCd in both the normal volunteer subjects and in the GERD patients is the same. Second, this intrinsic muscarinic smooth muscle component moves in lock step with the crural diaphragm during respiration in both the normal subjects and in the GERD patients. Thus, in both the normal and GERD groups the crural diaphragm is rigidly attached to this intrinsic muscarinic smooth muscle pressure component by the phrenoesophageal ligament. The distal intrinsic muscarinic smooth muscle pressure component (“lower” LES), is absent in 100% of the GERD patients evaluated in this study (FIG. 4).

The distal muscarinic pressure peak normally constitutes one third of the gastroesophageal junction high-pressure zone pressure profile as measured by the area under the curve in normal subjects. This pressure profile may be important to the anti reflux barrier, since it is the most distal component at the EGS and therefore the first line of defense against reflux of gastric contents into the esophagus in the resting state. From the normal volunteer data, this distal muscarinic pressure profile complex remains relatively stationary while the crural diaphragm and proximal intrinsic muscarinic smooth muscle pressure components move proximally about 2 cm during FE. Without the distal muscarinic pressure profile, the distal esophagus is unprotected and may be exposed to gastric pressure, increasing the probability of opening during the resting state.

### Example 3

### 002-D and 3-D Endoluminal Ultrasound Localization of Endoscopic Plications with Simultaneous Manometry (Location of Plications and Depth of Sutures)

The purpose of these experiments was to use 2-dimensional high-resolution endoluminal ultrasound with 3 dimensional reconstructions of ultrasound images in conjunction with simultaneous manometry to determine the physiologic effects of the endoscopic plications, to determine the locations of the plications with respect to the crural diaphragm (CD) and the suture depth.

Overview of methods. Ten patients with symptomatic GERD underwent simultaneous ultrasound and manometry pre EndoCinch. Eight of those patients responded to the EndoCinch procedure with a reduction or elimination of their symptoms. Seven of these patients underwent post EndoCinch evaluation with simultaneous ultrasound and manometry during breath holding under full inspiration (FI) and full expiration (FE) with a machine pull-through of a simultaneous US and manometry catheter assembly, at a velocity of 5 mm/s, from the stomach into the thoracic esophagus. Pull-throughs were repeated after intravenous administration of atropine. Simultaneous ultrasound and manometry was repeated at one month after endoscopic plication, with and without atropine. It was felt that one month was sufficient time to allow for the resolution of edema and inflammation due to placement of the plications.

For each pull-through the axial location of the lower margins of the right crural diaphragm (RCD) was located on the ultrasound image and used as a reference point. The upper margin of the crural diaphragm was also located on the ultrasound image. In addition the start of the pull through was located and used as a constant and invariant reference point. Pressure was referenced to intragastric pressure.

Subject Selection. Eight of the ten GERD study patients responded to the EndoCinch procedure with a reduction or elimination of their symptoms. Seven of these patients underwent pre and post EndoCinch evaluation with simultaneous ultrasound and manometry before and one month after the EndoCinch procedure 8. These seven patients (three male and four female) with GERD (33-66 Years, mean age 45±10.7 years) were evaluated over the same time period with the same procedure as the normal subjects.

The GERD patients all complained of heartburn and/or regurgitation prior to the EndoCinch procedure, which was relieved with high dose proton pump inhibitors. All subjects gave IRB approved informed consent to take part in the studies, and all subjects were tested in accordance with the policies of the National Institute of Health and Temple University School of Medicine. Exclusion criteria for all subjects included subjects on any medication, which could affect the gastroesophageal junction high-pressure zone. This included prokinetic agents, erythromycin type antibiotics and anticholinergics. The following medical conditions were also considered exclusion criteria: abdominal surgery involving the stomach or esophagus, diabetes, scleroderma, achalasia and current pregnancy.

Endoscopy. All study subjects underwent upper endoscopy using a Pentax 2900 video endoscope (Pentax, Orangeburg, N.Y., USA) using topical oral anesthesia with Cetacaine (Getylite Industries, Pennsauken, N.J., USA), with or without sedation. However, hiatal hernia was not an exclusion criterion in the GERD study group.

Endocinch procedure. The system used to place the plications is the Bard EndoCinch Suturing System. EndoCinch, a commercial system used to permit suturing in GI track, uses two flexible endoscopes. One endoscope is used to place the sutures and the other to secure the sutures in place. An overtube is placed into the esophagus over a savory dilator. The endoscope, with the EndoCinch suturing device, is placed through the overtube. A capsule, present at the end of the endoscope, is positioned against the tissue to be sutured. Suction is applied to bring the fold of tissue to be sutured into the capsule.

A needle is activated, which drives a suture, with a suture tag, through the tissue. Once the sutures are placed at the appropriate site, suction is turned off to release the fold of tissue and the endoscope is withdrawn from the esophagus. The suture tag is then reloaded, and the endoscope is reinserted to place a second stitch adjacent to the first one. A suture-securing device is actuated to cinch and cut the suture and a plication is formed. Three plications were placed in each patient in a spiral fashion starting at approximately 2 cm below the gastroesophageal junction and moving proximally up in a circumferential manner to just below the gastroesophageal junction. The simultaneous ultrasound and manometry procedures were repeated at approximately one month after the Endocinch procedure in order to allow any swelling and inflammation due to the procedure to subside.

Equipment. A custom assembly was constructed which combined a high frequency ultrasound (HFUS) transducer with a perfused manometer to simultaneously obtain GEJHPZ musculature cross-section images and corresponding pressures. A 20 Mhz ultrasonographic transducer was placed within a 6 French (3 mm outer diameter) catheter (Microvasive, Boston Scientic, Watertown Mass.). The transducer rotated at 15-30 Hz to provide 360 degrees esophageal cross-section imaging with 0.1 mm axial slice thickness and a typical penetration of about 2 cm. Images were recorded on VHS videotape on a Kay Elemetrics swallowing workstation (Kay-Elemetrics, N.J.). A second catheter was glued to the first catheter, 3 French (1.5 mm outer diameter) angiography catheter, which was used to perform perfused manometry at 250 Hz. A small side hole port was made on the second catheter at the same level as the ultrasound transducer. The manometry system fed into the Kay Elemetrics workstation providing temporal synchronization of the two data sources.

A custom pull-through machine provided a calibrated, constant retraction of the simultaneous ultrasound and manometry catheter in a proximal direction through the stomach and the esophagus at 0.5 cm/s. The pressure data were saved to a computer file and the HFUS images digitized into 256 gray level, 640×480 lossless TIFF files.

Procedure and data collection. Prior to insertion of the simultaneous ultrasound and manometry transducer assembly into the proximal stomach, the back of each subject's throat was numbed with Cetacaine and the nose numbed with Lidocaine to reduce discomfort during the catheter's passage through the nostril. An IV was prepared to allow for the later administration of atropine.

High-frequency endoscopic ultrasound (HFUS) was co-localized with manometric pressure in the 7 GERD patients with breath holding under Full Inspiration (FI) and Full Expiration (FE) during a machine pull-through of the catheter assembly at 5 mm/s from the stomach into the thoracic esophagus. The subject lay supine with his or her back at approximately a 35-degree angle. Subject movement was minimized during the duration of the study. HFUS imaging verified that the initial transducer position was in the proximal stomach at both full inspiration (FI) and full expiration (FE). After ensuring the catheter's position was correct, the catheter was marked at the nares to ensure accurate repositioning of the transducer assembly in the stomach at the PTS reference location after withdrawal from the GEJHPZ.

Pull-throughs were repeated after injection and intravenous administration of atropine. HFUS images were recorded at 30 f/s and manometry at 250 Hz. For each pull-through the axial locations of the distal margin of the right crus muscle was quantified. Pressure was referenced to intragastric pressure.

The spatial references used in this study were the distal margin of the right leaf of the right crus (RCS), and the initiation of the pull-through (PTS). All analysis was carried out with in-house computer software, image pro plus software or Volview software.

Each subject had multiple insertions of the transducer assembly into the stomach and subsequent data collection during a constant speed retraction with the pull-through machine. Each of these cases is defined as a “pull through”. Each pull through was performed in one of two respiratory states. The subject was instructed to inhale as deeply as possible and hold his or her breath for the FI pull-throughs. For FE pull-throughs, the subject was instructed to exhale as far as possible and hold his or her breath. Each pull through began in the stomach, at the pre-marked level of the transducer (PTS) and ended after the transducer had traveled into the distal esophagus. Three pull throughs were collected for the FI and FE respiratory states:

After collection of the pre-atropine data, 0.6 mg of atropine was administered intravenously. During the remainder of the study, atropine was given as a continuous IV infusion at 0.25 mg/hr. After waiting roughly 30 minutes to assure abolishment of the smooth muscle contribution of the IS, three more pull-throughs were collected for the same positions and respiratory states.

Data Analysis. Data analysis was performed in a blinded fashion from the FI, FE, FI post-atropine, and FE post-atropine pull-throughs for the pre and post EndoCinch subjects. Only the “best” quality HFUS data was used, which was determined subjectively, by the quality of the ultrasound images (CD).

After data collection, the gastric baseline pressures were determined by examining the pressure signal prior to the start of each pull through (CD). All pressures were reported as referenced to gastric pressure.

The axial extent of the three plications in each patient, were mapped on a graph of the pressure vs. length. In each patient the three plications were localized on the three dimensional ultrasound image and the length of the plications were drawn against the pressure profile and referenced to the crural diaphragm.

Spatial References. The beginning and end of the RCd was determined (LM) and independently checked for accuracy (CD) on HFUS. The length of the crural diaphragm was also used as a spatial reference as measured on HFUS.

The PTS (Pull through Start) was defined as the location of the transducer assembly in the stomach at the time that the pull through began. This position was determined by the mark on the catheter made with a Sharpie pen at the nares. The catheter was repositioned using this mark to ensure that the catheter was in the same and proper location before the start of each pull through.

Statistics. Ensemble plots are presented. Data analysis included the percent contribution of the three components of the HPZ. All of the above data was combined in order to differentiate the intrinsic pressure profiles, including the pressure generated by the plications, from the crural diaphragm contributions to the GEJHPZ and to determine the effect of respiration on the position and pressure relationships of the intrinsic LES and CD as components of the GEJHPZ.

Three Dimensional Reconstruction. Ultrasound images were stored on videotape for later reconstruction. The video output from a VCR was connected to a dedicated 3D system (Volview) for acquisition, post-processing, and visualization of the 3D images. A 20 second scan length contains 600 images. Immediately after acquisition, the data was reviewed for adequacy of the 3D formats.

The 3D image is displayed as a polyhedron representing the boundaries of the reconstructed volume. Using an intuitive interface with 3D cues, the user can rotate the polyhedron and rapidly obtain the desired cut planes that display the anatomic information in any orientation (e.g., transverse, longitudinal, coronal, or oblique). Pressure data was correlated with individual 2D.

Imaging of the suture material in an in vitro water bath showed that the echo characteristic of the suture material is hyperechoic. A bright dot appeared on the ultrasound image when the suture material was imaged in cross section and a hyperechoic line appeared on the ultrasound image when the suture material was imaged in longitudinal section.

Analysis of normal control subjects. It was shown that the GEJHPZ subtraction curves between the pre- and post-atropine pressures are formed from two pressure peaks in both FI and FE, (roughly 1.5-1.6 cm apart). In FI, the proximal peak due to the physiologic upper “lower” esophageal sphincter overlaps and extends above the ES while the lower peak due to the gastric sling/clasp muscle fibers straddles the ES. In FE, the ES and the proximal pressure peak shifts superiorly relative to the distal pressure peak.

During FI the proximal intrinsic component (LES) makes up approximately 66% of the intrinsic pressure profile and 25% of the entire pressure profile by analysis of area under the curve. The distal intrinsic component (gastric sling/clasp muscle fibers) made up approximately 34% of the intrinsic pressure profile and 13% of the entire pressure profile and the crural diaphragm (ES) made up approximately 62% of the entire pressure profile (IS plus ES). In normal volunteers during FE the proximal intrinsic component (LES) makes up approximately 69% of the intrinsic pressure profile and 43% of the entire pressure profile (IS plus ES). The distal intrinsic component made up approximately 31% or the intrinsic pressure profile and 20% of the entire pressure profile and the crural (ES) diaphragm made up approximately 39% of the entire pressure profile (IS plus ES).

Analysis of GERD patients pre Endocinch. Two of the GERD patients were found to have hiatal hernia at endoscopy (one small and one moderate sized hiatal hernia). The other five GERD patients showed no sign of hiatal hernia. The pre minus the post atropine subtraction curves in the GERD patients demonstrated distinctly different pressure profiles from the subtraction curves in the normal volunteer subjects in both full inspiration and full expiration. In the GERD patients the subtraction curve between the pre- and post-atropine pressure curves demonstrated that the distal intrinsic muscarinic smooth muscle pressure peak (lower LES), which was present in the normal volunteer subjects was absent in both the inspiratory and expiratory phases in all the GERD patients, while the proximal intrinsic muscarinic cholinergic smooth muscle pressure peak (upper LES) seen previously in the normal volunteer subjects was present and at the same axial position relative to the RCd as in the normal volunteer subjects.

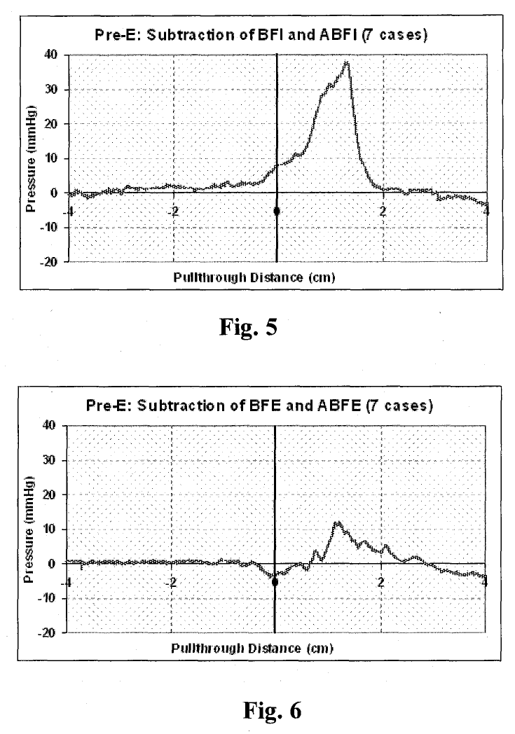

In the GERD group, the width of the gastroesophageal junction high-pressure zone during FI was 2.5+/−0.7 cm and the width during FE was 2.5+/−0.7 cm (p=0.9). Unlike the normal volunteer subjects, there was no significant change in the width of the high-pressure zone between FI and FE. The width of the CD during FI was 2.1+/−0.8 and during FE was 2.1+/−0.6 (p=0.9) (FIGS. 5 and 6). Like the normal control subjects, the width of the CD did not change between FI and FE. These results indicate that there was no lengthening of the gastroesophageal junction high pressure zone from FI to FE in the GERD patients as opposed to the normal volunteer subjects.

The beginning of the RCd moved 1.4 cm proximally relative to the initiation of the pull-through position between FI and FE; the intrinsic muscarinic cholinergic smooth muscle pressure profile moved approximately the same distance in concert with the CD. This is also approximately the same distance that the RCd moved between FI and FE in the normal volunteer subjects.

Pre Endocinch, during FI the proximal intrinsic component (LES) makes up 100% of the intrinsic pressure profile and 62.9% of the entire pressure profile (IS plus ES). The distal intrinsic component gastric sling/clasp muscle fibers made up 0% of the intrinsic pressure profile and 0% of the entire pressure profile and the crural diaphragm (ES) made up approximately 37.1% of the entire pressure profile (IS plus ES) by analyzing the area under the curve. During FE the proximal intrinsic component (LES) makes up 100% of the intrinsic pressure profile and 26% of the entire pressure profile (IS plus ES). The distal intrinsic component (gastric sling/clasp muscle fibers) made up 0% or the intrinsic pressure profile and 0% of the entire pressure profile (IS plus ES) and the crural diaphragm (ES) made up approximately 74% of the entire pressure profile (IS plus ES).

Analysis of GERD patients post Endocinch. Eight of the ten patients (80%) had significant clinical benefit after endoscopic-plication. In the seven patients that underwent post Endocinch simultaneous ultrasound and manometry studies, the pressure profile more closely resembled the pressure profile in the normal control subjects than the prior pressure profile pre Endocinch, in that there was a distinct pressure curve just below the GEJ, similar to the pressure curve generated by the gastric sling/clasp muscle fibers. Although the location of the distal pressure peak was the same, the magnitude and width of the distal pressure peak was greater than in the normal volunteers. The magnitude of the proximal pressure peak, representing the LES, remained constant and in the same location relative to the crural diaphragm as in the pre Endocinch studies.

During FI the proximal intrinsic component (LES) makes up 54% of the intrinsic pressure profile and 21.5% of the entire pressure profile (IS plus ES). The distal intrinsic plication made up 46% of the intrinsic pressure profile and 24.7% of the entire pressure profile and the crural diaphragm (ES) made up approximately 37.1% of the entire pressure profile (IS plus ES) by analysis the area under the curve. During FE the proximal intrinsic component (LES) makes up 52% of the intrinsic pressure profile and 23.4% of the entire pressure profile (IS plus ES). The distal intrinsic component (endoscopic plication) makes up 48% or the intrinsic pressure profile and 25.2% of the entire pressure profile (IS plus ES) and the crural diaphragm (ES) makes up is approximately 74% of the entire pressure profile (IS plus ES).

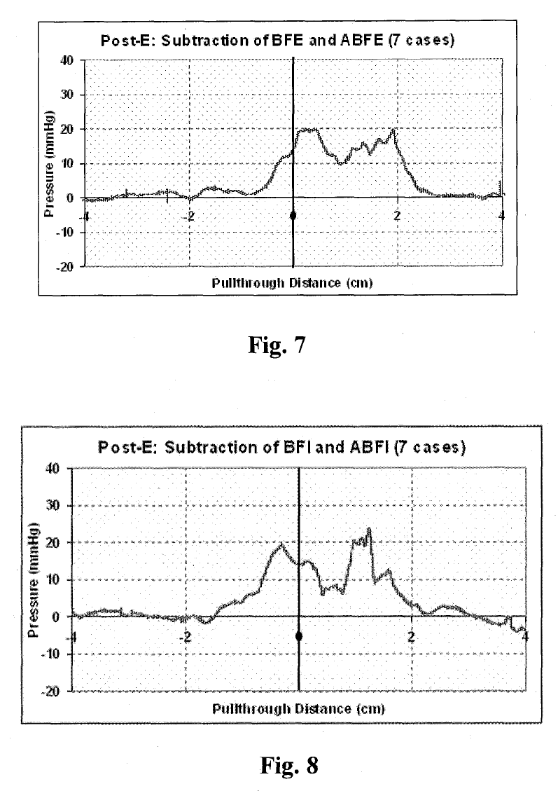

The mean width of the entire high pressure zone during FI was 3.7 cm. The width during FE was 3.6 cm. There was no significant change in the width between FI and FE. The width of the placation in FI was 2.0 cm and in FE is 1.65 cm. The peak pressure due to the plication was 19.3 mmHg in FI and 19.6 mmHg in FE. The width of the proximal muscarinic pressure profile was 1.47 cm in FI and 1.25 cm in FE. The peak pressure of the proximal muscarinic pressure profile was 23.8 mmHg in FI and 19.4 mmHg in FE. The distance between the peak of the plication and the peak of the proximal muscarinic pressure profile was 1.49 cm in FI and 1.6 cm in FE (FIGS. 7 and 8). The width of the high pressure pre vs post endoscopic plication increased by 48% in FI (p<0.001) and by 44% in FE (p<0.001).

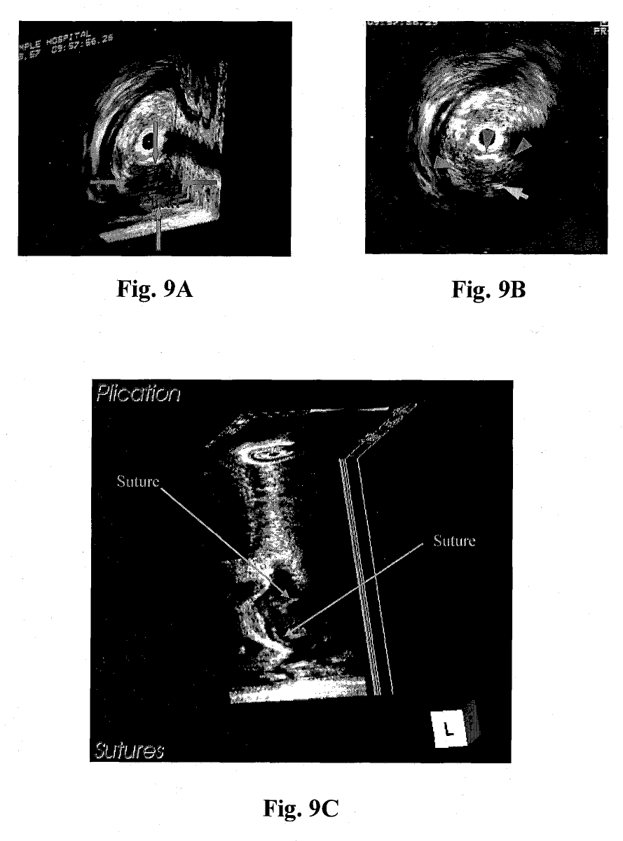

Imaging. On 2D ultrasound imaging the plications appear as hypoechoic round structures (FIG. 9 A-C). The plications appear as hypoechoic spherical structures on 3D US. The sutures appear as hyperechoic dots when imaged in cross section on 2 D ultrasound images and as hyperechoic lines within the plications when imaged longitudinally on 2D ultrasound images. The sutures appear as hyperechoic lines within the plications on the 3D ultrasound images. The majority of sutures are localized to the submucosa.

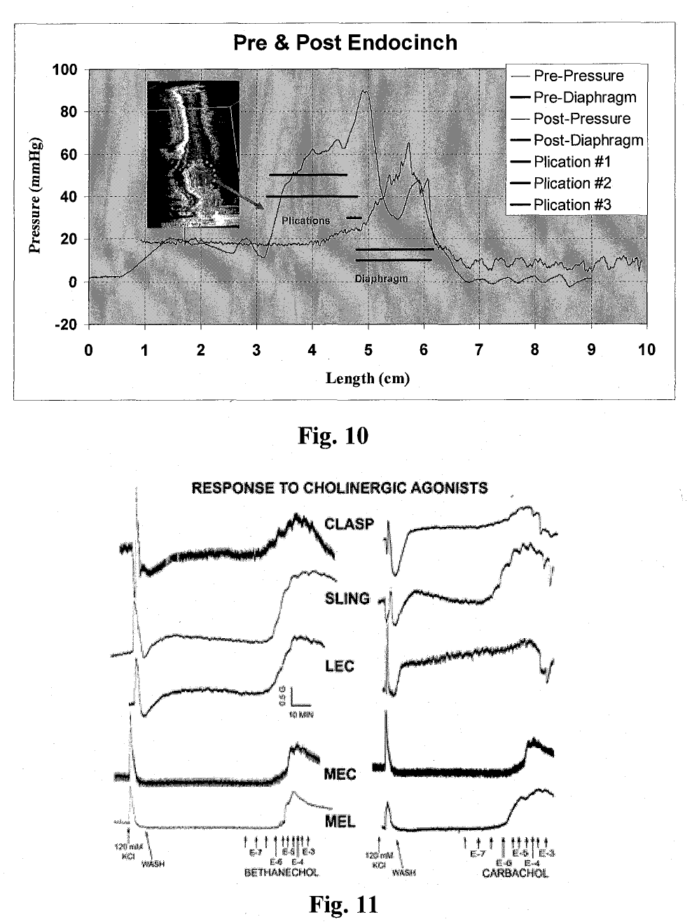

FIG. 10 shows the area of the three plications which are mapped out on a graph of the pressure vs the length. The majority of the plications were located at or just below the Right Crus of the diaphragm. The distal portion of the pressure profile in the GEJHPZ was increased and lengthened in the area of the plications. The area in which the plications were localized showed a dramatic increase in the length of the high pressure zone, the absolute pressure in the area of the plications and the area under the pressure curve compared to the pre EndoCinch pressure curves.

This picture shows the pressure profiles through the gastroesophageal junction high pressure zone pre- and post-EndoCinch in a patient with GERD, referenced to the right crural diaphragm. Note that the dark curve represents the pre-EndoCinch pressure profile, while the white curve represents the post-EndoCinch pressure profile. The horizontal lines represent the axial locations of endoscopic plications as imaged on ultrasound (above the dark curve), and the right crural diaphragm (top line between 5 and 6 cm, below the curves (post-EndoCinch), and bottom line between 5 and 6 cm below the curves (pre-EndoCinch)).

Comparing Pressures with the Location of plications. All the sutures are located in the mucosa and the submucosa in this patient. There are no sutures in the muscularis propria. All the plications are located below the diaphragm on the ultrasound images. There is a significant increase in pressure post Endocinch in the distal esophagus and proximal stomach which correlates with the location of the three plications.

Discussion. Simultaneous ultrasound and manometry was developed to study the physiology (pressure) and anatomy (muscle thickness) of the esophagus in order to better understand esophageal mechanics. In previously performed studies, simultaneous ultrasound/manometry was employed, to establish the relative roles of the intrinsic lower esophageal sphincter (LES) and the crural diaphragm (CD) in normal subjects and in patients with GERD. Simultaneous endoluminal ultrasound and manometry was utilized to differentiate the intrinsic LES from the crural diaphragm contributions to the GEJHPZ using pharmacologic manipulation, since the esophageal smooth muscle of the IS is muscarinic and can be abolished with atropine, leaving the striated muscle of the ES unaffected. Under the assumption that the GEJHPZ is a superposition of the pressures generated by the intrinsic and extrinsic sphincters, it is possible to extract the intrinsic sphincter pressure profile by subtracting the extrinsic sphincter pressure curve from the overall GEJHPZ pressure when properly referenced. Pressure from other sources or residual muscarinic pressure is removed in the subtraction process. This subtraction was performed for each individual subject referenced to RCd and subsequently averaged across subjects.

It was determined that the GEJHPZ was composed of three overlapping pressure profiles from the upper intrinsic muscarinic LEC muscle, the lower intrinsic muscarinic gastric clasp/sling muscle fiber complex and the non-muscarinic skeletal muscle crural diaphragm.

The relative physiologic contribution of each component was determined and it was found that GERD patients lack the pressure profile consistent with the lower intrinsic muscarinic gastric clasp/sling muscle fiber complex. It was hypothesized that the lack of this pressure profile due to the gastric sling/clasp muscle fibers allows exposure of the distal esophagus to gastric contents and is a possible underlying pathophysiologic abnormality leading to gastroesophageal reflux disease.

It was determined that the use of endoscopic plication was able to correct the pathophysiologic abnormality (lack of gastric clasp/sling muscle fiber complex pressure profile) by reestablishing a distal high pressure zone in the area of the missing distal pressure profile and lengthening the HPZ.

In the current study, the high-pressure zone of the distal esophagus and the area of endoscopic plications in the same patients with GERD were evaluated before and after endoscopic plication. In addition, 2D ultrasound images were reconstructed in 3D along with corresponding pressure profiles. This allows the dynamics of the HPZ and the endoscopic plications to be viewed in a unique manner so that the physiology of the pressure profile can be evaluated simply and intuitively with the three dimensional ultrasound images. The location of the plications, the depth and location of the sutures, the configuration of the plications, and the relationship of the plications to changes in the pressure profile were evaluated.

The distal pressure profile was reestablished at the same location as the gastric clasp/sling pressure profile in normal volunteers after endoscopic plication. The endoscopic plications appear as hypoechoic round structures on 2D US and hypoechoic spherical structures on 3D US. The sutures appear as hyperechoic dots or lines within the plications. The sutures were localized to the submucosa. The plications were located at or just below the Right Crus of the diaphragm. The distal portion of the pressure profile in the GEJHPZ was increased and lengthened in the area of the plications and the placement and length of the plications correlated exactly with the increase in the distal pressure profile and the area under the curve of the pressure graphs.

Simultaneous ultrasound and manometry with 3D reconstruction of ultrasound images allows the detailed analysis and correlation of the anatomic and physiologic changes, which occur after endoscopic plications to treat GERD. 2D and 3D US with simultaneous manometry can be used to localize endoscopic plications, determine the depth and location of the sutures, and determine changes in the pressure profile pre- and post-Endocinch.

### Example 4

### Comparison of the Neurotransmitters and Receptors Responsible for In-Vitro Contraction and Relaxation of Smooth Muscle Strips from Whole Gastro Esophageal Specimens Obtained from Organ Transplant Donors with and without GERD

Preliminary Experiments. Twenty nine normal whole human stomach and esophagus specimens were obtained from organ procurement agencies (The International Institute for the Advancement of Medicine and the National Disease Research Interchange). The specimens were obtained from brain dead patients maintained on life support who consented to organ transplant donation and their next of kin consented to donation of non-transplantable organs for research. Because only two of these patients had a history of GERD and one of these had a 360° fundoplication, no direct comparisons of results can be made between specimens from GERD patients and specimens from organ donors without GERD.

The specimens were harvested within 30 minutes after cross clamping the aorta. The stomach contents were gently rinsed out with saline. The esophageal and pyloric openings were ligated and the entire specimen was transported to the laboratory on ice by overnight courier immersed in either University of Wisconsin (Beltzer's Viaspan) organ transport media (UW) or HTK which is composed (in mM) of: NaCl—15, KCl—9, Potassium hydrogen 2-ketoglutarate—1, MgCl₂—4, histidine NaCl—18, tryptophan—2, mannitol—30, CaCl₂.2H₂O—0.015. Smooth muscle strips for contractility studies obtained from these specimens were found to be viable for at least 3 days after harvesting if the specimen is continually maintained immersed in ice cold transport media.

The specimens were dissected in a cold room (0-5° C.). The greater and lesser omentum were removed. The outermost longitudinal fibers descending from the esophagus across the stomach were individually removed by sharp dissection. This exposed the sling fibers as a U shaped group of fibers approximately 8 mm wide enveloping the esophagus around the greater curvature of the stomach and the semicircular clasp fibers around the lesser curvature opposite to the cardiac notch.

The clasp fibers are oriented perpendicular to the sling fibers and connect between the open ends of the U shaped sling fiber complex suggesting that the sling/clasp fiber complex may function similar to a Bolero tie to cinch closed the esophageal opening and prevent reflux of stomach contents back up the esophagus.

Beginning at the cardiac notch, the sling fiber complex was separated from the underlying submucosa by sharp dissection and this tissue plane was followed completely around the lesser curvature thus separating the clasp fibers from underlying submucosa. The clasp fiber complex was removed from the sling fiber complex by sharp dissection and cut into 10-12 smooth muscle strips approximately 3×3×8 mm with the long axis parallel to the direction of the muscle fibers. Similar muscle strips were cut from the middle of the sling fiber complex such that these strips were derived from the sling fibers in the cardiac notch. These smooth muscle strips were suspended in 10 ml muscle baths between platinum electrodes in Tyrode's solution continuously bubbled with 95% O₂/5% CO₂ and maintained at 37° C. The muscle strips were stretched to approximately 150% of their slack length which produced approximately 1 gram of basal tension and were then allowed to accommodate to the muscle bath for at least 60 minutes prior to investigation of contractile response.

Representative traces of the contractile response to 120 mM KCl in isotonic Tyrode's solution followed by a wash and 60 minute re-equilibration period then a cumulative concentration response curve to the selective muscarinic receptor agonist bethanechol (left) or the muscarinic and nicotinic agonist carbachol (right) is shown in FIG. 11. These strips were prepared from gastric clasp fibers, gastric sling fibers, the esophageal circular fibers approximately 1-2 cm proximal to the transitional zone (LEC), mid esophageal circular fibers taken 7 cm proximal to the transitional zone (MEC) and esophageal longitudinal fibers taken 10 cm proximal to the transitional zone (MEL). As can be seen there are striking differences in both the KCl and cholinergic responses of these different human gastroesophageal smooth muscle dissections.

Membrane depolarization with 120 mM KCl causes an initial relaxation followed by a contraction in clasp fibers but induces a contraction in the other fibers. The sling fibers produce the greatest cholinergic contractile response followed by the clasp and longitudinal fibers (MEL) while the circular fibers from the mid (MEC) or lower esophagus (LEC) produce the lowest tension in response to bethanechol or carbachol. In all 5 dissections the response to increasing concentrations of the muscarinic agonist bethanechol increases to a point and then the tension remains constant or fades slightly with increasing bethanechol concentrations.

The carbachol response is qualitatively quite different between the different dissections. In clasp, sling and LEC fibers, the contractile response increases with increasing carbachol concentrations then an abrupt relaxation response is observed at 300 μM and with each successive higher carbachol exposure. This is not observed in MEC or MEL fibers as shown in FIG. 11 nor in circular muscle fibers dissected from the human stomach antrum, fundus or pylorus. Indeed we do not observe this relaxation response to high concentrations of carbachol in any other smooth muscle preparations including human urinary bladder as well as rat, guinea pig, rabbit, canine, feline or porcine urinary bladder nor guinea pig gallbladder or rabbit uterus. Thus this relaxation response to high concentrations of carbachol is unique to the smooth muscle fibers in the GEJ.

Another difference between the clasp fibers and the circular or longitudinal fibers from the mid esophagus was found to be the relaxant response to high concentrations of carbachol (FIG. 11, right panel). These relaxations are observed, but to a lesser percentage of the maximal contractile response, in muscle fibers from sling or LEC. Carbachol activates both muscarinic and nicotinic receptors. The sling, clasp and LEC fibers were found to relax in response to nicotine after being stimulated to contract maximally with the selective muscarinic receptor agonist bethanechol. These nicotinic relaxations are unique to these specific smooth muscle components of the gastro-esophageal junction and are not observed in smooth muscle strips from other parts of the esophagus or stomach or other visceral smooth muscle including bladder or uterus from multiple species. These nicotinic receptor induced relaxations in bethanechol pre-contracted muscle fibers may be an in vitro correlate of the in vivo TLESR that are responsible for GERD.

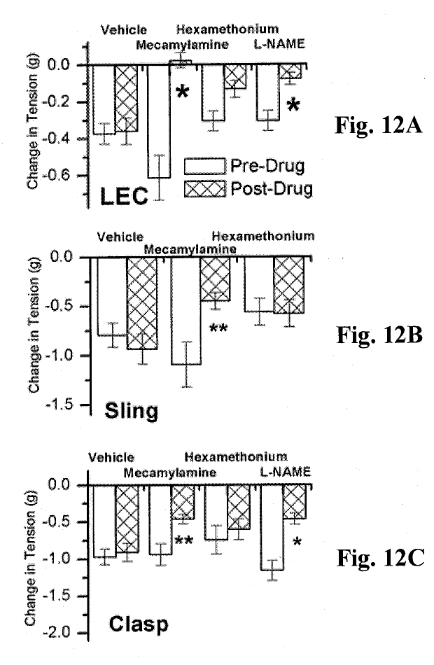

Clasp, sling and LEC muscle strips were exposed to increasing concentrations of carbachol then after repeated washings over a 60 minute re-equilibration period, strips were exposed to different potential inhibitors of relaxation for 30 minutes then rechallenged with increasing concentrations of carbachol. Results are shown in FIG. 12 for the neuronal nicotinic receptor antagonists mecamylamine (10 μM) and hexamethonium (100 μM) and the nitric oxide synthase (NOS) inhibitor L-NAME (50 μM). Results are expressed as mean±sem for at least 8 strips from at least 3 different organ donors for the maximal decrease in tension from the maximal contractile response. Because sling fibers respond to carbachol with a greater contractile response than the clasp or LEC fibers, the relaxation response to high carbachol concentration is less as a percentage of the maximal contractile response in sling fibers (29±4%) than in clasp (131±6%) or LEC fibers (54±9%). While mecamylamine inhibited the carbachol induced relaxations, hexamethonium did not. L-NAME inhibited relaxations in clasp and LEC fibers and has not yet been tested in sling fibers.

Because mecamylamine blocks the carbachol induced relaxation but hexamethonium does not, this may indicate that the nicotinic receptors mediating this relaxation response may be unique and thus a potential target for development of selective agents that block this unique receptor without affecting other nicotinic receptor mediated effects at other sites throughout the body.

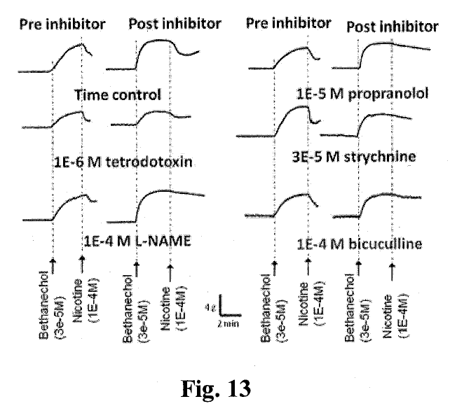

Similar preliminary studies were recently performed looking for drugs that will prevent the relaxations induced by nicotine after stimulating contractions with the selective muscarinic receptor agonist bethanechol. Strips were exposed to a maximally effective concentration of bethanechol (30 μM) then, after reaching maximal tension, 100 μM nicotine was added. After a 60 minute repeated washing and re-equilibration period, strips were exposed to potential inhibitors of relaxations for 30 minutes then rechallenged with bethanechol followed by nicotine. Representative traces from these experiments in sling fibers from two different organ donors is shown in FIG. 13. As can be seen, 100 μM nicotine induces a relaxation after the strips contracted maximally to 30 μM bethanechol and this relaxation is reasonably reproducible comparing the first and second challenge in the time control strip. Preventing neuronal action potentials with the sodium channel blocker tetrodotoxin reduced the relaxation but not completely. Interestingly, the NOS inhibitor L-NAME, the β adrenergic receptor antagonist propranolol, the glycine receptor antagonist strychnine and the GABA_{A} antagonist bicuculline all inhibited the nicotine induced relaxations. Similar responses were found to for clasp and LEC fibers.

### Example 5

### Comparison of Contractile Response of Gastric Sling/Clasp Fibers from GERD and Healthy Subjects

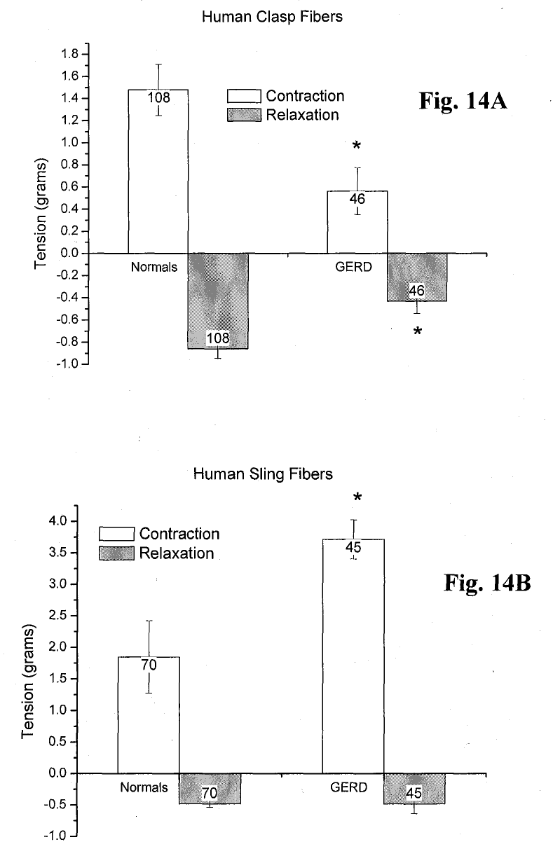

The maximal carbachol induced contraction and relaxation were measured in is human clasp and sling fibers obtained from brain dead organ donors. Differences between donors with GERD compared to donors without GERD were seen in both the contractile and relaxation response to the mixed muscarinic and nicotinic receptor agonist carbachol in both clasp and sling fibers (FIG. 14). The maximal carbachol induced contraction is significantly lower (p<0.05) in clasp fibers from donors with GERD (n=46 muscle strips from 3 individual donors) compared to non-GERD donors (n=108 muscle strips from 8 individual donors). The reverse is seen in sling fibers, the maximal carbachol induced contraction is greater (p<0.05) in sling fibers from donors with GERD (n=45 muscle strips from 2 individual donors) compared to the donors without GERD (n=70 muscle strips from 4 individual donors). The carbachol-induced relaxations seen with high concentrations of carbachol are significantly lower (p<0.05) in clasp fibers from donors with GERD (n=46 muscle strips from 3 individual donors) compared to non-GERD controls (n=108 muscle strips from 8 individual donors). There is no difference in the carbachol induced relaxation in sling fibers between GERD (n=45 muscle strips from 2 individual donors) and non-GERD controls

These studies show that there are three separate pressure components at the GEJHPZ in normal volunteer subjects, two of which are intrinsic muscarinic pressure components. The upper intrinsic muscarinic pressure components is the LEC sphincter muscle and the lower intrinsic muscarinic pressure components, the gastric sling/clasp muscle fiber complex. In GERD patients the lower pressure component is missing. This study, based on muscle contraction studies, indicates that the gastric clasp muscle fibers contract significantly less in patients with GERD than in individuals without GERD. Thus, the missing distal intrinsic cholinergic pressure component in GERD patients is presumably due to a decrease in the tonic contraction of the gastric clasp fibers because of decreased responsiveness to muscarinic stimulation or increased relaxation because of increased responsiveness to nicotinic stimulation. Interestingly, endoscopic plication appears to work by replacing the lower pressure component.

However, both the muscle of the gastric sling/clasp muscle fiber complex and it's neural innervations are somewhat intact after endoscopic plication, despite the lack of pressure generated by this complex in patients with GERD before the EndoCinch procedure. This was demonstrated by showing that the pressure profile, which is generated by placement of the plications, is abolished by the effects of atropine. A possible explanation for the pathophysiology of the missing lower intrinsic muscarinic pressure profile is that the gastric sling muscle fibers do not oppose the gastric clasp muscle fibers due to a diminished tone within the gastric clasp muscle fibers prior to the EndoCinch procedure. This leads to a laxity in the area of the cardia of the stomach, which causes a slight gap at the GEJ. This anatomic abnormality is easily seen endoscopically in the setting of a hiatal hernia, but is more difficult to visualize endoscopically in patients with GERD but no hiatal hernia.

The fact that the sutures of the plications do not actually penetrate into the muscularis propria suggests that the sutures located within the mucosa and submucosal complex, immediately above or medial to the clasp and sling muscle fibers, act to pull the two muscle groups into apposition. In so doing, the area in which the plications are placed becomes stiffer. Thus this area, the cardia of the stomach, becomes more resistant to distension, both during normal swallowing, and during TLESR induced by increased gastric volume and pressure. TLESRs are initiated by stretch receptors in the cardia, and the cardia is easier to distend or stretch in patients with GERD due to the missing gastric sling/clasp pressure profile, patients with GERD.

### Example 6

### Gastric Clasp and Lower Esophageal Circular Muscle Fibers from GERD Patients with Barrett's Esophagitis have a Decreased Contractile Response to Cholinergic Stimulation

Methods. Stomach and esophagi were obtained from 17 human transplant donors: 11 with no GERD history, 4 with probable GERD (proton pump inhibitor use) and 2 with definite GERD (Barrett's esophagitis). The contractile response to increasing carbachol concentrations was determined. Muscarinic receptor density was measured by subtype selective immunoprecipitation of [3]H-QNB binding.

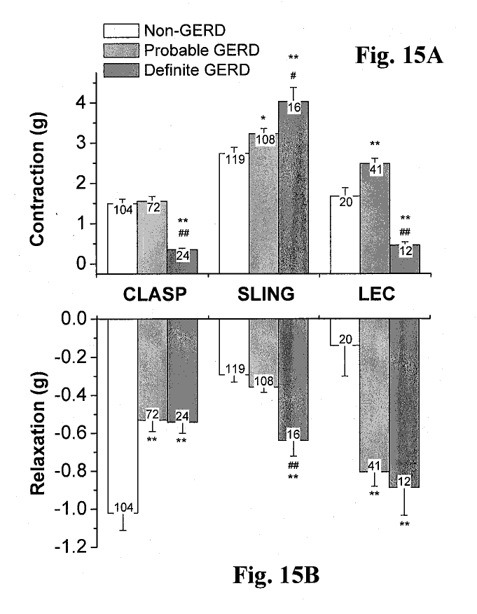

Results. Clasp and LEC fibers from definite GERD have decreased maximal contractile response (FIG. 15). Contractility is increased in sling fibers of both definite and probable GERD (FIG. 15). Concentrations of carbachol higher than 100 μM induce relaxations that are decreased in clasp fibers of probable and definite GERD. Relaxations are greater in sling fibers from definite GERD and higher in LEC from probable and definite GERD. Total and M₂ receptor density is statistically lower in the GERD than non-GERD specimens in both clasp and sling fibers and M₃ density is statistically lower in GERD than non-GERD clasp fibers.

This suggests that a myogenic defect in clasp fibers may be involved in GERD pathophysiology in the organ donors with Barrett's esophagitis. The greater contractile response in the sling fibers from GERD donors suggest a possible compensatory mechanism for the lack of contractility of the clasp muscle fibers.

### Example 7

### M₂ and M₃ Muscarinic Receptor Mediated Contractions of Human Gastroesophageal Smooth Muscle

Materials. All drugs and chemicals were obtained from Sigma Chemical Company (St. Louis, Mo.) except darifenacin (which was a generous gift from Pfizer Limited, Sandwich, Kent), digitonin (Wako Pure Chemical Company, Osaka) and pansorbin (Calbiochem, La Jolla, Calif.).

Human stomachs with the attached esophagus were obtained, with consent, from brain dead organ transplant donors through either the National Disease Research Interchange or the International Institute for the Advancement of Medicine. Peritoneal fat was removed and dissection began using micro-scissors to remove the most superficial longitudinal fibers in a circular pattern around the esophagus. The deeper circular fibers were removed next, moving from the greater curvature towards the lesser curvature. The exact location of the sling and clasp fibers were identified at the greater and lesser curvature of GEJ, respectively, once the superficial longitudinal fibers were removed. Sling muscle fibers were removed from a relatively straight section of the greater curvature. Clasp fibers were obtained 2-3 cm distal to GEJ along the lesser curvature. The LEC fibers were obtained from the thickened area of the esophagus approximately 1-2 cm proximal to the stomach. The MEC and MEL fibers were obtained from the esophagus 10 cm proximal to the stomach. The muscles were further divided into individual strips, each measuring 1-2 mm in width and 8-10 mm in length. Care was taken to ensure the orientation of the muscle fibers parallel to the muscle strips. The muscle strips were then suspended with 0.5 g of tension in tissue baths containing 10 ml of modified Tyrode's solution (125 mM NaCl, 2.7 mM KCl, 0.4 mM NaH₂PO₄, 1.8 mM CaCl₂, 0.5 mM MgCl₂, 23.8 mM NaHCO₃, and 5.6 mM glucose) and equilibrated with 95/5% O₂/CO₂ at 37° C.

Bethanechol Response Curves. Following equilibration to the bath solution for 30 minutes, the strips were incubated for 30 minutes in the presence or absence of one of 3 concentrations of the competitive M₂ selective antagonist methoctramine (0.1, 1 or 10 μM) or the competitive M₃ selective antagonist darifenacin (10, 30, or 100 nM). Dose response curves were derived from the peak tension developed following the cumulative addition of non-subtype selective muscarinic receptor agonist bethanechol (10 nM to 10 mM final bath concentration). Either vehicle or one concentration of methoctramine or darifenacin was used for each muscle strip. Dose ratios were determined based on the average of the responses of vehicle (H₂O) treated strips. EC₅₀ values were determined for each strip using a sigmoidal curve fit of the data (Origin, Originlab Corp. Northampton, Mass.) and Schild plot's were constructed.

Immunoprecipitation. Immunoprecipitation of muscarinic receptors from the individual dissections was performed with subtype specific antibodies as previously described (Braverman A S et al. (2007) Neurology & Urodynamics, 26:63-70). Briefly, the tissues were homogenized at 100 mg/ml in cold Tris EDTA buffer (TE) with 10 μg/mL of the following protease inhibitors: soybean and lima bean trypsin inhibitors, aprotinin, leupeptin, pepstatin, and α2-macroglobulin. 20 μL of the non-subtype selective muscarinic receptor antagonist [3H] QNB (49 Curies/mM, approximately 4,000 cpm/μL) per mL assay homogenate was added and incubated at room temperature for 30 minutes with inversion every 5 minutes. Samples were pelleted via centrifugation at 20,000 g for 10 minutes at 4° C. and the pellet was solubilized in TE buffer containing 1% digitonin and 0.2% cholic acid (1% TEDC) with the above protease inhibitors at 100 mg wet weight per ml. Samples were incubated for 50 minutes at 4° C., with inversion every 5 minutes then centrifuged at 30,000 g for 45 minutes at 4° C. The supernatant containing the solubilized receptors was incubated overnight after addition of the M₂ or M₃ antibody, or vehicle at 4° C.

To determine total receptor density, samples were desalted over Sephadex G-50 minicolumms with 0.1% TEDC. M₂ and M₃ receptors were precipitated by adding 200 μL pansorbin, and incubated at 4° C. for 50 minutes, with inversion every 5 minutes. The precipitated receptors were pelleted via centrifugation at 15,000 g for one minute at 4° C. and the pellet was surface washed with 500 μL of 0.1% TEDC. 50 μL of 72.5 mM deoxycholate/750 mM NaOH was added and incubated for 30 minutes at room temperature. The pellet was resuspended in 1 mL of TE buffer and neutralized with 50 μL of 1M HCl. Radioactive counts were determined by liquid scintillation spectrometry. Protein content was determined by a Coomassie blue dye binding protein assay using bovine serum albumin as a standard. Receptor density (mean±SEM) is reported as femtomoles (fmoles) receptor per mg solubilized protein.

Statistics. All statistical differences were determined by a nonparametric statistic (Wilcoxon Rank Sum/Mann-Whitney U-test) because of non-homogenous variances. Results.

Immunoprecipitation. Five different dissections of human gastroesophageal smooth muscle were studied. These sections were clasp, sling, LEC, MEL and MEC. For each dissection we determined total, M_{Z} and M₃ muscarinic receptor densities using immunoprecipitation and did this as a prelude to subsequent studies of bethanechol-induced contraction which are also described below. The results of the receptor density determinations are shown in table 3. The rank order of total receptor density in the 5 different smooth muscle dissections was sling>LEC>clasp>MEL>MEC fibers. The M₂ receptor subtype density followed a similar pattern as total receptor density with sling>clasp>LEC>MEC>MEL fibers. However the M₃ receptor subtype density was 60-83 fmol/mg protein for the sling, LEC, MEC and LEC fibers but approximately 10 fold less (8±2 fmol/mg protein) for the clasp fibers.

TABLE 3
Total, M₂ and M₃ muscarinic receptor density (fmoles/mg solubilized
protein,) for different dissections of human GEJ muscles.

| Muscle | Total | M₂ | M₃ | M₂/M₃ ratio |
|---|---|---|---|---|
| Clasp | 228 ± 20 b | 116 ± 16 c d e | 8 ± 2 b c d e | 14.5 |
| Sling | 357 ± 7 c d e | 171 ± 6 c d e | 60 ± 14 | 2.85 |
| LEC | 244 ± 12 d e | 73 ± 7 | 83 ± 13 | 0.88 |
| MEC | 190 ± 7 | 59 ± 9 | 69 ± 9 | 0.86 |
| MEL | 209 ± 10 | 54 ± 4 | 78 ± 3 | 0.69 |

Total muscarinic receptor density was determined by total [3H] QNB binding, while M₂ an M₃ receptor density was determined using subtype selective immunoprecipitation. Results are reported as mean ± SEM for at least duplicate determinations from 2 individual organs for clasp and sling fibers, while n = 3 donors for LEC, MEL and MEC fibers. b = significantly different from sling fibers, c = significantly different from LEC fibers, d = significantly different from MEC fibers, e = significantly different from MEL fibers. p < 0.05 if not bold, p < 0.01 if bold. Statistical differences were determined using nonparametric statistics with a Mann-Whitney U-test.

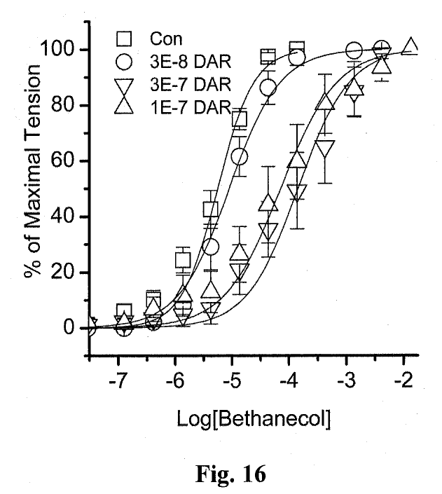

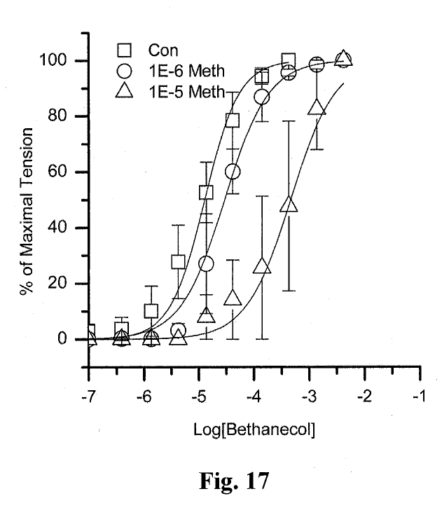

Concentration-effect relationships. Each muscle section was studied for isometric tension development in response to bethanechol and each demonstrated a dose-related response to this agonist. For example, FIG. 16 shows the graded concentration-effect relationship for bethanechol in clasp fibers. Also shown in this figure are the curves for graded doses of this agonist with three different fixed concentrations of darifenacin, a relatively selective M₃ competitive antagonist. Shown in FIG. 17 are the curves for graded doses of this agonist with no antagonist and with two different fixed concentrations of methoctramine, a relatively selective M₂ competitive antagonist. The fitted curves show an obvious dose dependency and, further, they also show dextral shifts resulting from each antagonist dose. While these log plots show approximate parallelism (indicative of competitive inhibition), the relatively low potency of darifenacin (pA2=7.8±0.2) suggests that M₂ receptors mediate contraction, while the low potency of methoctramine (pA2=6.3±0.2) suggests that M₃ receptors mediate contraction in human clasp fibers. Darifenacin potency (pA2) is 8.0±0.1, 8.2±0.2, 8.2±0.1 and 8.4±0.2 and methoctramine potency (pA2) is 6.8±0.2, 6.2±0.2, 5.7±0.2, and 5.6±0.3 in sling, LEC, MEC, and MEL fibers respectively.

TABLE 4
Table 4. Maximal tension and bethanechol potency determined
for the different dissections of human GEJ muscles.

| Muscle | BETH MAX | BETH pEC₅₀ |
|---|---|---|
| Clasp | 1.20 ± 0.17 (n = 14) b d | 5.8 ± 0.09 (n = 14) d |
| Sling | 2.18 ± 0.24 (n = 37) c d e | 4.98 ± 0.10 (n = 37) c d |
| LEC | 0.92 ± 0.09 (n = 29) | 5.19 ± 0.11 (n = 29) d |
| MEC | 0.79 ± 0.07 (n = 24) e | 4.34 ± 0.08 (n = 24) e |
| MEL | 1.37 ± 0.21 (n = 10) | 4.80 ± 0.09 (n = 10) |

Results are reported as mean ± SEM. b = significantly different from sling fibers, c = significantly different from LEC fibers, d = significantly different from MEC fibers, e = significantly different from MEL fibers. p < 0.05 if not bold, p < 0.01 if bold. Statistical differences were determined using nonparametric statistics with a Mann-Whitney U-test.

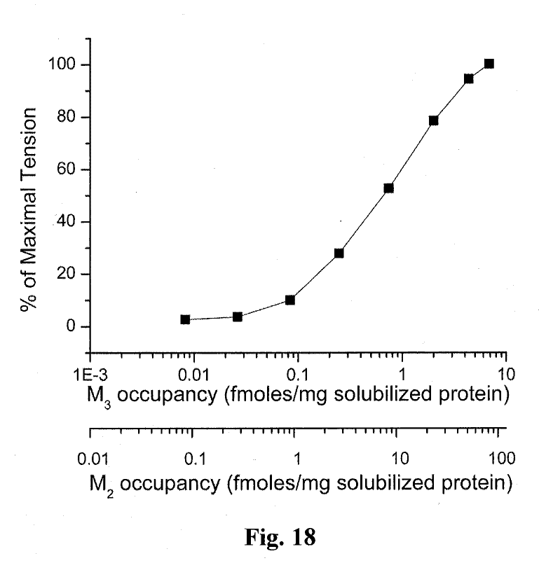

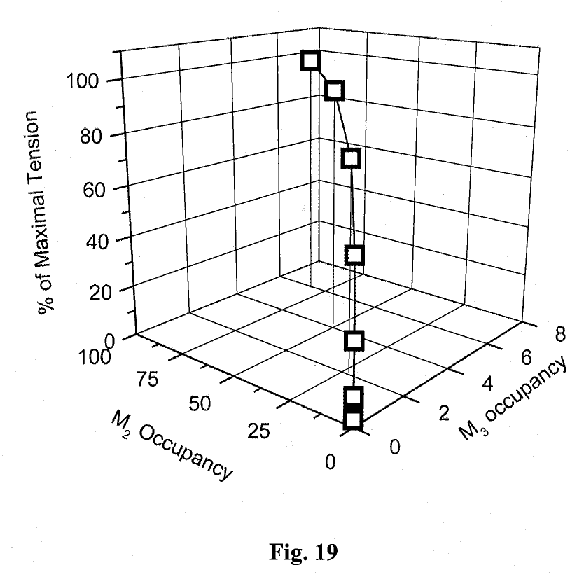

These potencies in clasp and sling fibers suggest that the bethanechol effect is mediated by both M₂ and M₃ receptors; hence, using Schild plot analysis which is based on the assumption that one receptor is mediating the effect is inappropriate. For that reason, and to add clarity to the relative contribution of each receptor subtype, each bethanechol concentration was transformed to receptor occupations of both M₂ and M₃ receptors. That transformation was based on mass-action binding which, at equilibrium, gives receptor occupation=[A][R]/([A]+K_{A}), where [A] denotes the agonist concentration, [R] is the receptor concentration and K_{A} is the agonist dissociation constant (reciprocal of affinity). For this purpose published values of K_{A} were used (Evans T et al. (1985) Biochem. 3. 232:751-757; McKinney M et al. (1991) Mol. Pharmacol. 40:1014-1022) for bethanechol as follows: K_{A} for M₂=170 μM and K_{A} for M₃=110 μM. The concentration-effect curve in clasp fibers is shown FIG. 18 in which the abscissa scales show the simultaneous values of M₂ and M₃ occupancy that follow from the bethanechol concentrations that were employed. It is noted that the M₂, M₃ occupation pair that gives 50% of the maximum tension is the pair (8.8, 0.9). However, from this graph it is not apparent that occupancy of both M₂ and M₃ receptors occurs simultaneously resulting in contraction. This critical point is more clearly evident in an alternative view of this dual receptor occupation-effect (FIG. 19) which is a three dimensional plot with the effect shown as the height above the M₂-M₃ occupation plane.

Antagonist Effects. The presence of a fixed concentration of a competitive antagonist reduces the agonist occupancy to a lower quantity given by the equilibrium equation of Gaddum:

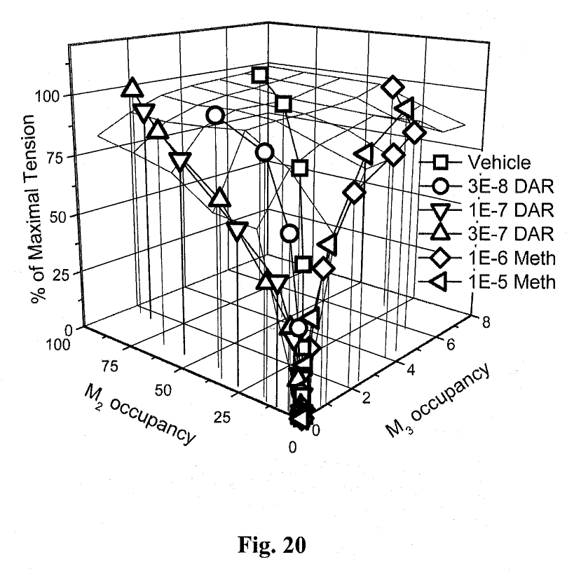

receptor occupation=[A][R]/[A]+K_{A}(1+[B]/K_{B})

where [B] is the antagonist concentration and K_{B} is its dissociation constant. Of course, this holds at each receptor with each receptor's applicable values of [R], K_{A} and K_{B}. Thus, the presence of the antagonist yields bethanechol occupancy at M₂ and M₃, each calculated from the above, thereby giving a view of occupation of this receptor pair and its corresponding effect. This relation is shown in the three dimensional plot (FIG. 20).

This graph, for clasp fibers, was generated using published affinity values (Caulfield M P (1993) Pharmacol. & Therapeutics 58:319-379; Caulfield M P et al. (1998) Pharmacol. Revs. 50:279-290), from three different doses of darifenacin (pKBM₃=8.65, pKBM₂=7.2, thus relatively selective for M₃) and two different doses of methoctramine (PKBM₃=6.6, pKBM₂=8.1, thus relatively selective for M₂). The use of the two antagonists in several different fixed concentrations yielded an array of M₂, M₃ occupancy values and their associated effects.

A more global view of these results is provided in the form of a response surface, also shown in FIG. 20, which shows that both M₂ and M₃ receptors have a significant role in mediating contraction in clasp fibers. This is based on the occupancy-effect relationship in the presence of the antagonists. In the presence of darifenacin, where very few M₃ receptors are occupied by bethanechol, the occupancy-effect relationship is more dependent on M₂ occupancy than on M₃ occupancy. This can be seen on the surface plot in FIG. 20 where the occupancy effect curve in the presence of darifenacin is almost parallel with the axis of M₂ occupancy and shows very little deflection along the M₃ occupancy axis. In contrast, in the presence of methoctramine, where very few M₂ receptors are occupied by bethanechol, the occupancy effect relationship is more dependent on M3 occupancy than on M₂ occupancy.

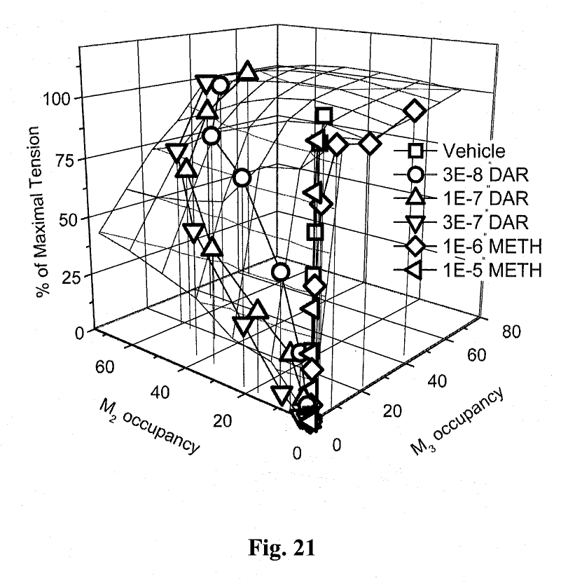

Other Gastrointestinal Muscle Fibers. The analysis of occupancy-effect relations described above for the clasp fibers was also conducted on the human sling, LEC, MEC and MEL smooth muscle fibers. For each muscle group a surface plot, similar to that of the clasp fibers, was generated. The surface plot for sling fibers (not shown), which have more M₂ receptors than M₃ receptors (table 3) is similar to the surface plot for clasp fibers which also have more M₂ than M₃ receptors. The surface plot for LEC fibers which have more M₃ receptors than M₂ receptors has a different shape (FIG. 21). The surface plots for MEC and MEL fibers, which also have more M₃ receptors than M₂ receptors, are similar to that for LEC fibers (not shown). In these muscle groups, the occupation-effect relationships demonstrate that contraction is more dependent on M₃ occupation than M₂ receptor occupation. This is demonstrated by the occupation-effect relationship of the LEC fibers shown in FIG. 21.

When the M₂ selective antagonist methoctramine is present, the occupation-effect relationship shows that contraction is dependent on occupation of M₃ receptors. In addition, in the presence of darifenacin, contraction increases with increasing M₂ occupancy, but only up to a point, maximal tension is only obtained when the bethanechol concentration is high enough to compete for occupation of the M₃ receptors. This is demonstrated by the deflection to the right (increasing M₃ occupancy) of the occupation effect curve in the presence of darifenacin (FIG. 21).

### Example 8

### Comparison of Muscarinic Receptor Subtypes Mediating Contraction of Human and Pig Gastric Clasp and Sling Fibers

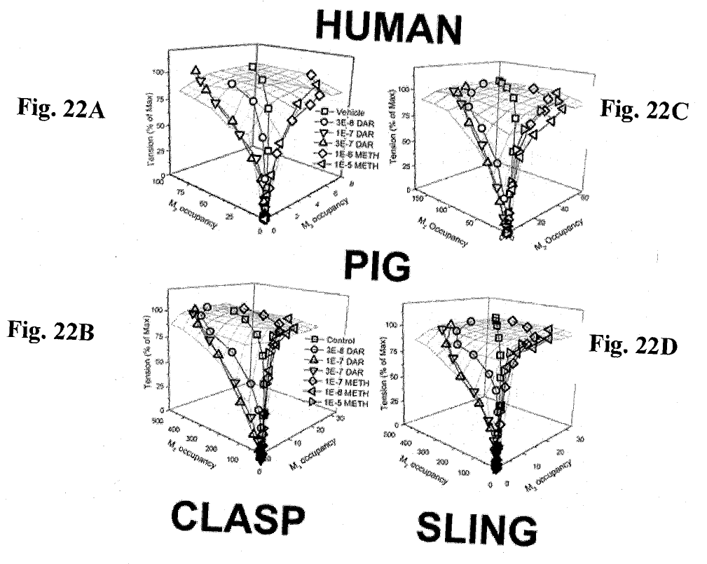

This study determined how closely the contractile physiology of the pig gastroesophageal junction follows the human. Human tissue was obtained from organ transplant donors, and pig tissue from a slaughterhouse. Total M₂ and M₃ receptor density was determined by subtype specific immunoprecipitation. Total and M₂ were observed to be higher in pig than human. M₃ receptors were observed 2 fold higher in human than pig sling and over 2 fold lower in human than pig clasp fibers (Table 5). The methoctramine and darifenacin potency to inhibit bethanechol contractions, calculated by classic Schild analysis, indicates that both M₂ and M₃ receptors cause contraction which violates the assumption of one receptor causing the effect. An analysis method relating dual occupation of M₂ and M₃ receptors to the contractile response was developed based on the published Ka values of M₂ and M₃ receptors for bethanechol of 170 μM and 110 μM respectively, and mass-action binding which, at equilibrium, gives receptor occupation=[A][R]/([A]+Ka), where [A] denotes the agonist concentration, [R] is the receptor concentration and Ka is the agonist dissociation constant (reciprocal of affinity). Three dimensional plots for M₂ and M₃ occupation and contractile response are shown in FIG. 22A-D. Although the M₃ receptor subtype density is different between human and pig, the physiology of the contractile response is similar. This indicates that the pig may be a good model for human gastroesophageal junction physiology.

TABLE 5
Muscarinic Receptor Density (fMol/mg soluble protein)

| | Total Human | Total Pig | M₂ human | M₂ Pig | M₃ human | M₃ pig |
|---|---|---|---|---|---|---|
| Clasp | 228 ± 20 | 797 ± 14 | 116 ± 16 | 512 ± 17 | 8 ± 2 | 20 ± 1.3 |
| Sling | 353 ± 7 | 856 ± 27 | 171 ± 6 | 443 ± 39 | 60 ± 14 | 32 ± 1.5 |

### Example 9

### Pharmacologic Specificity of Nicotinic Receptors Mediating Relaxation of Muscarinic Receptor Pre-Contracted Human and Pig Gastric Clasp Fibers

The circular muscle fibers at the end of the esophagus are traditionally considered the lower esophageal sphincter. However, the first barrier to gastric reflux is actually the clasp/sling fiber complex of the stomach. Relaxation mediates transient lower esophageal sphincter relaxation underlying the pathophysiology of gastroesophageal reflux disease (GERD). This study determined the pharmacologic specificity of the nicotinic receptor mediated relaxation of pre-contracted strips of human and porcine clasp muscle fibers.

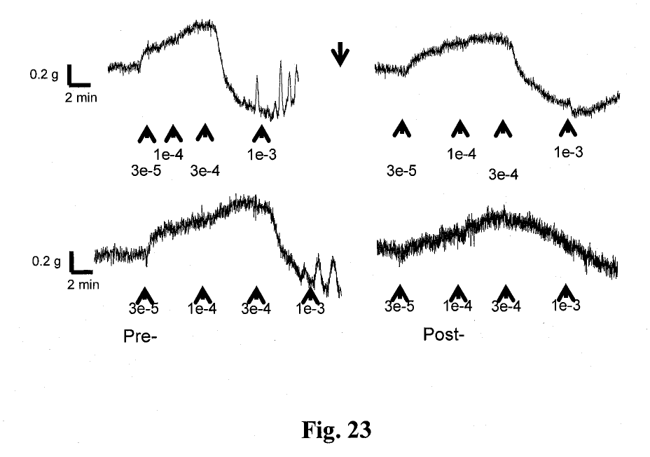

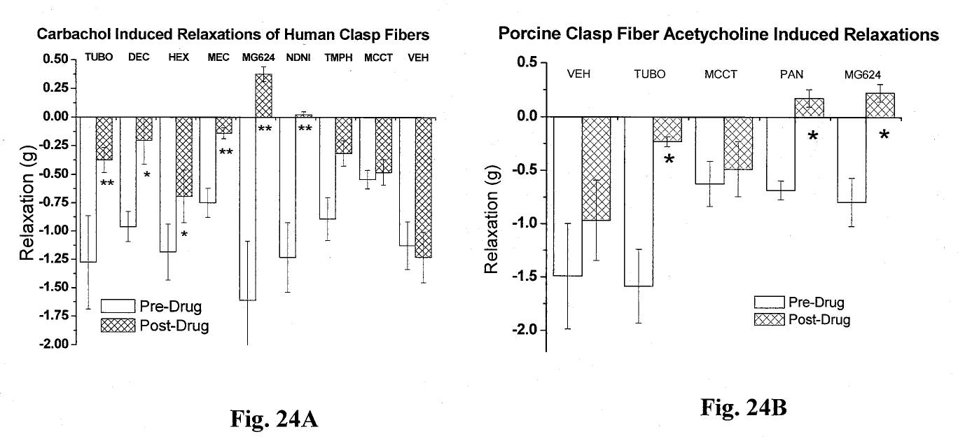

Human specimens were obtained from organ transplant donors. Clasp fiber muscle strips were exposed to increasing carbachol concentrations (human) or acetylcholine with physostigmine to block cholinesterase (pig). At concentrations higher than 30 uM, abrupt relaxations were produced (FIG. 23). After 60 minutes of repeated washing, strips were exposed to various ganglionic and neuromuscular nicotinic receptor antagonists for 30 minutes then rechallenged with cholinergic agonists. Results, as a percentage of relaxation in time control strips are shown in FIG. 24 and Table 6.

TABLE 6
Nicotinic Receptor antagonist used in the study with their classic
specificity and the number of organs and muscle strips studied.

| | | | Human | | Human | | Pig | |
|---|---|---|---|---|---|---|---|---|
| | | | Clasp | | Sling | | Clasp | |
| Agent | Specificity | [Molar] | organs | strips | organs | strips | organs | strips |
| Vehicle (VEH) | None | | 7 | 31 | 4 | 23 | 2 | 9 |
| Hexamethonium | Ganglionic | 1E−4 | 4 | 16 | 3 | 16 | | |
| (HEX) | | | | | | | | |
| Mecamylamine | Ganglionic | 1E−5 | 4 | 11 | 3 | 15 | | |
| (MECA) | | | | | | | | |
| Decamethonium | Neuromuscular | 3E−4 | 3 | 14 | 3 | 15 | | |
| (DECA) | | | | | | | | |
| Tubocurarine | Neuromuscular | 1E−5 | 3 | 11 | 3 | 12 | 1 | 5 |
| (TUBO) | | | | | | | | |
| MG624 | Ganglionic | 1E−4 | 1 | 4 | | | 1 | 4 |
| NDNI | Ganglionic | 1E−4 | 1 | 4 | | | | |
| TMPH | Ganglionic | 3E−5 | 1 | 5 | | | | |
| Methylylcaconitine | α7 | 1E−7 | 1 | 5 | | | 1 | 4 |
| (MCCT) | | | | | | | | |
| Pancoronium (PAN) | Neuromuscular | 1E−5 | | | | | 1 | 4 |

Carbachol induced relaxations of human clasp fibers is inhibited by the neuromuscular nicotinic blockers tubocurarine and decamethoniunn. In addition, the ganglionic blockers hexamethonium, mecamylamine, MG624 and NDNI blocked the relaxations. The alpha 7 specific antagonist methyllycaconotine and TMPH (alpha 3, alpha 4, beta 2 and beta 4) were ineffective.

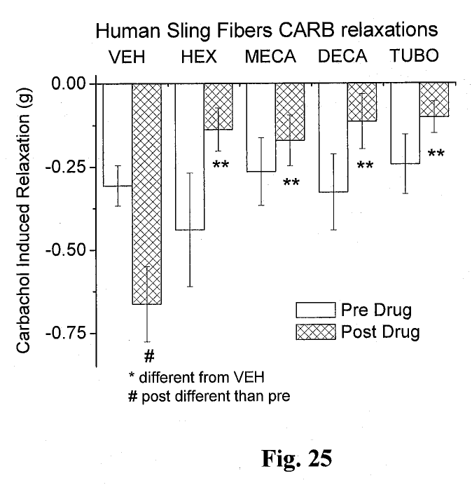

The neuromuscular blockers d-tubocurarine, decamethonium (human) and pancuronium (pig) inhibited relaxation whereas the ganglionic blocker hexamethonium (human) and the alpha7 nicotinic receptor subunit selective antagonist methyllycaconitine did not (FIG. 25). Other ganglionic blockers such as MG624, NDNI and TMPH blocked these relaxations whereas mecamylamine was only partially effective in human tissue.

Human sling fibers do not relax as much as clasp fibers, and both ganglionic (HEX and MEC) and neuromuscular (TUBO and DECA) nicotinic receptor antagonist inhibit relaxations. The results of this study indicate that the pharmacology of the nicotinic receptors mediating relaxation of the gastric clasp and sling muscle fibers may be unique and a potential target for development of selective agents for the treatment of GERD.

### Example 10

### PCR-Identification of Unique NAcR Subunits in Gastroesophageal Fibers

Total RNA was isolated from approx. 100 mg of clasp, sling, LEC, MEC, and MEL fibers from 4-5 individual specimens using Absolutely RNA® mini-prep kit (Stratagene, Inc., La Jolla, Calif.) according to the manufacturer's instructions. Following quantitation, 2 μg of total RNA was reverse-transcribed using the SuperScript™ II Reverse Transcriptase Kit (Invitrogen). Each resulting cDNA sample was diluted 1:50 and 4 μl was analyzed by quantitative PCR using a BioRad MyiQ™ instrument and SYBR green detection. Cycling conditions included a 95 degrees C. melting step for 10 seconds, followed by a 58 degrees C. annealing/detection step for 45 seconds, for 50 cycles. The cycle in which the fluorescence increased significantly above background (threshold cycle) was determined in duplicate for each of the nicotinic receptor subunits (alpha 1, 2, 3, 4, 5, 6, 7, 9 and 10, and beta 2, 3, and 4), with beta actin as a positive control.

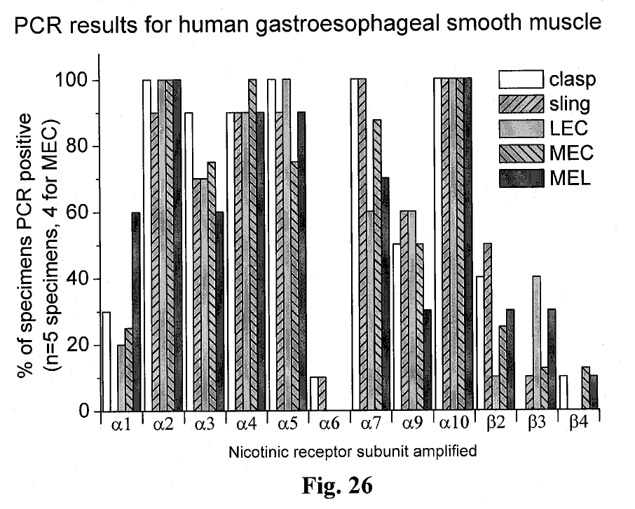

A tissue was considered positive for a subunit if either of the duplicate determinations had a threshold cycle of less than 45. Results shown in FIG. 26 are displayed as the percentage of positive specimens for each of the subunits in each of the 5 different smooth muscle dissections. In general, few of the specimens were PCR positive for the alpha 1 and 6 or beta 4 subunits. Most specimens were positive for the alpha 2, 3, 4, 5, 6, 7, 9, and 10 subunits.

### Example 11

### Immunohistochemical Localization of Nicotinic Receptor Subunits Present in the Human Gastric Sling/Clasp Muscle Fiber Complex

Gastric clasp muscle fibers are defective in GERD patients (attenuated contractile and relaxation response to carbachol). The relaxation is mediated by nicotinic receptors-pentameric, ligand gated ion channels. Current experiments aim to determine which nicotinic receptor subunits are present on which cell types in human gastric clasp muscle fibers.

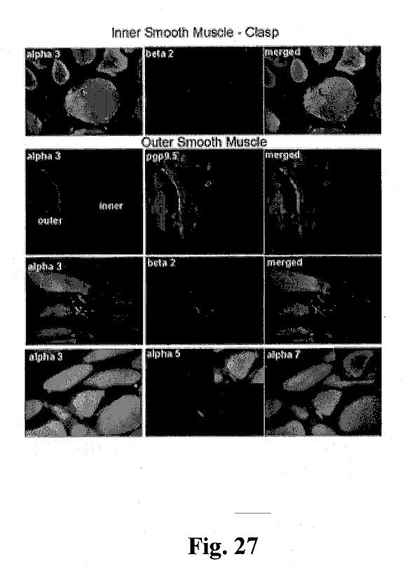

Human stomach and esophagus from organ donors were fixed in 4% paraformaldehyde, frozen sectioned and stained using specific antibodies for nicotinic receptor α subunits 2, 3, 5, 7, 9, and 10 and β2. Co-localization was investigated, as was dual or triple labeling with antibodies to cellular markers for nerves (PGP9.5), smooth muscle (smoothelin), and interstitial cells of Cajal (ICC; CD117) (FIG. 27). Axons are positive for nicotinic receptor subunits α3, 5, 7, 9 and β2, but α5 and 9 are only on axon terminals and patch-like sites on smooth muscle fibers. Subunits α3 and 7 co-localize in axonal processes and terminals, while β2 co-localizes with α3, 7 and 9 in axonal terminals and patches. In smooth muscle, α3, 5, 7 and β2 staining was observed in surface patches, a subset of which co-localize with axonal staining. β2 co-localizes with α5 and 7 in smooth muscle. ICCs, small CD117 positive cells intermingled with smooth muscle fibers. These show co-localization of β2 with α2, 3, 5, 7, 9 and 10 (FIG. 27). Thus, several nicotinic receptor subunits are expressed in human clasp fibers. While many subunits are expressed in smooth muscle, ICC, and nerves, each cell type expresses a different combination.

### Example 12

### Pharmacologic Specificity of Nicotinic Receptor Mediated Relaxation of Muscarinic Receptor Pre-Contracted Human Gastric Clasp and Sling Muscle Fibers

Relaxation of the gastro-esophageal high pressure zone, including gastric clasp and sling fibers, is involved in the transient lower esophageal sphincter relaxations (TLESRs) involved in GERD pathophysiology. The gastric sling/clasp muscle complex does not contribute to the high pressure zone in GERD patients indicating a role in pathophysiology.

The aim of this study is to identify drugs that might prevent nicotine induced relaxation of muscarinic receptor pre-contracted gastric clasp and sling fibers.

Methods. Human gastric muscle strips obtained from 4 organ donors were contracted to 30 μM bethanechol then relaxed with 100 μM nicotine. After wash and adding inhibitors, strips were rechallenged. Relaxation inhibitors included: the sodium channel poison tetrodotoxin (TTX, 1 μM), the nitric oxide (NO) synthase inhibitor L-NAME (100 μM), the β adrenoceptor antagonist propranolol (10 μM), the glycine receptor antagonist strychnine (30 μM) and the GABA_{A} receptor antagonist bicuculline (100 μM).

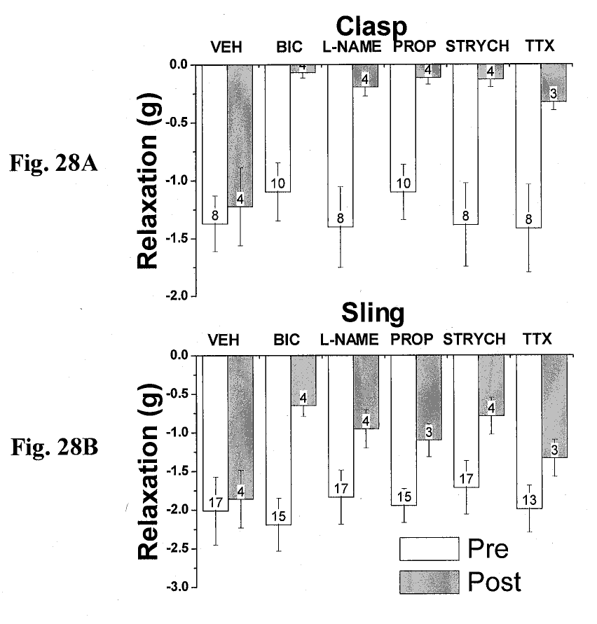

Results. As shown in FIG. 28, all inhibitors were more effective in preventing relaxation of clasp than sling fibers. Following muscarinic receptor stimulation, nicotinic receptor activation appears to cause release of multiple substances that relax clasp and sling smooth muscles including nitric oxide, norepinephrine acting on β adrenoceptors, GABA acting on GABA_{A} receptors and glycine acting on glycine receptors. Identification of agents that selectively prevent relaxations may be useful therapeutic agents to treat GERD by preventing TLESRs.

### Example 13

### Pharmacologic Classification of the Nicotinic Receptors Mediating Nicotine Induced Relaxation in Clasp, Sling, and LEC Fibers

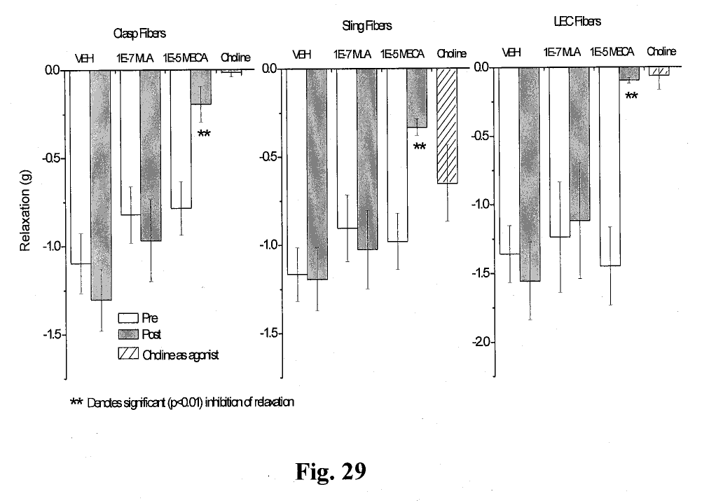

Clasp, sling, and LEC muscle strips were suspended in muscle baths and pre-contracted with 30 μM of the muscarinic receptor agonist bethanechol, then induced to relax with either 1 mM nicotine or 10 mM choline. Following extensive washing, the muscle fibers were exposed to either vehicle, 10 nM methyllycaconitine (MLA) or 10 μM mecamylamine (MECA), then re-exposed to bethanechol and nicotine. As can be seen in FIG. 29, MLA had no inhibitory effect while MECA significantly inhibited the nicotine induced relaxation in clasp, sling, and LEC muscle fibers. In addition, choline induced a significant relaxation in sling fibers, but not in clasp or LEC fibers. This data was generated to classify the nicotinic receptor subtype which mediates relaxation according to the nomenclature in Table 2 in Albuquerque, E. X., et al., Mammalian nicotinic acetylcholine receptors: from structure to function, Physiological Reviews, 2009. 89(1): pp. 73-120. In this classification, type 1A receptors (α7 homomers) are inhibited by MLA and but not MECA and choline is a full agonist. The above results rule out type 1A nicotinic receptors mediating relaxation in clasp, sling and LEC fibers. Type II nicotinic receptors (α4β2) are insensitive to MLA and choline is not an agonist. Type III receptors (α3β4β2) are not inhibited by MLA, but are inhibited by MECA and choline is a partial agonist. Type IV receptors (α2β4/α4β4) are not inhibited by MLA. Because MLA does not inhibit nicotine induced relaxation in clasp and LEC fibers while MECA does and choline is not an agonist, the nicotinic receptors mediating nicotine induced relaxation in clasp and LEC fibers has a pharmacologic profile consistent with either type II or type IV receptors. Because choline is an agonist while MLA does not inhibit nicotine induced relaxation in sling fibers while MECA does, the nicotinic receptor mediating relaxation in sling fibers has a pharmacologic profile consistent with either type III or type IV receptors. The actual subtype of nicotinic receptor mediating relaxation in clasp, sling and LEC fibers may be unique and may not be any of types described above.

### Example 14

### Nicotinic Subunit Receptor Staining in Clasp Fibers

A nicotinic subunit receptor staining of clasp fibers, carried out according to the protocol described in Example 11, is summarized in the paragraphs below and in Tables 7 and 8.

Axonal co-localization: Nerves stain positive for alpha 2, 3, 5, 7, 9 and beta 2. Alpha 3 and 7 staining is present consistently in axonal processes and terminals. All others are on specific regions of axons only: axonal terminals and patch-like sites on smooth muscle fibers. Beta-2 co-localizes in nerve terminals and patch-like sites on smooth muscles with alpha 2, 3, 7 and 9. Nerves stain negative for alpha 5 and 10.

Smooth muscle co-localization: Smooth muscles stain positive for alpha 3, 5, 7 and beta 2 in patchy sites on surface. A subset of these patches do not co-localize with axons. Beta-2 co-localizes with alpha 7 and alpha 5 and beta 2 in smooth muscle. Smooth muscle stains negative for alpha 2, 9 and 10.

Interstitial cells of Cajal co-localization: Beta 2 and alpha 2, 3, 5, 7, 9 and 10 staining is present in interstitial cells of Cajal that are present adjacent to smooth muscle fibers and in extracellular connective tissues. Beta 2 co-localizes with each.

Subunit co-localization: Beta-2 co-localizes in nerve terminals and patch-like sites on smooth muscles with alpha 2, 3, 7, 9 and 10. Beta-2 co-localizes with alpha 7 and alpha 5 and beta 2 in smooth muscle. Beta 2 co-localizes with each alpha subunit in interstitial cells of Cajal.

Other: Macrophages (CD11b+) and T cells (CD45+) stain negative for alpha 3, 7 (others not yet assessed). Mucosal glands stain positive for alpha 3, 5, 7 and beta 2. Mucosal glands contain co-localized alpha 7-beta 2, alpha 3-alpha5, alpha 5-alpha7, and alpha 3-alpha7. Vascular endothelium stains positive for alpha 2, 3, 5, 7 and beta 2. Alpha 3-beta 2, alpha 5-alpha 7, alpha 2-beta 2, alpha 3-alpha5, and alpha 3-alpha 7 co-localize in endothelium of blood vessels.

TABLE 7
Co-localization of subunits in Clasp Fibers.

| | | Smooth | Interstitial | | | | | | |
|---|---|---|---|---|---|---|---|---|---|
| | Axonal | Muscle | Cells of Cajal | | | | | | |
| | (PGP9.5+) | (Smoothelin+) | (CD117+) | alpha 3 | alpha 5 | alpha 7 | alpha 9 | alpha 10 | beta 2 |
| alpha 2 | + (on subset | − | + | | | | | | + |
| | of terminals) | | | | | | | | |
| alpha 3 | + | + (Patchy | + | X | | + | | | + |
| | | areas) | | | | | | | |
| alpha 5 | + (on subset | + (Patchy | + | + | X | | | | + |
| | of terminals) | areas) | | | | | | | |
| alpha 7 | + | + (Patchy | + | + | + | X | | | + |
| | | areas) | | | | | | | |
| alpha 9 | + (on subset | − | + | | | | X | | + |
| | of terminals) | | | | | | | | |
| alpha 10 | − | − | + and | | | | | X | + |
| | | | processes to | | | | | | |
| | | | muscles | | | | | | |
| beta 2 | + (on subset | + (Patchy | + | + | + | + | | | X |
| | of terminals) | areas) | | | | | | | |

TABLE 8
Co-localization of subunits in Sling Fibers.

| | | Smooth | Interstitial | | | | | | |
|---|---|---|---|---|---|---|---|---|---|
| | Axonal | Muscle | Cells of Cajal | | | | | | |
| | (PGP9.5+) | (Smoothelin+) | (CD117+) | alpha 3 | alpha 5 | alpha 7 | alpha 9 | alpha 10 | beta 2 |
| alpha 2 | − | − | | | | | | | + |
| alpha 3 | + | + | | X | | + | | | + |
| alpha 5 | − | + | | + | X | | | | + |
| alpha 7 | + | + | | + | + | X | | | + |
| alpha 9 | + | − | | | | | X | | + |
| alpha 10 | − | + | | | | | | X | + |
| beta 2 | + (in specific | + | | + | + | + | | | X |
| | areas) | | | | | | | | |

The above pharmacologic data is consistent with type II or type IV nicotinic receptors mediating relaxation in clasp and LEC fibers. Type II receptors are composed of α4β2 subunits and the immunohistochemistry results confirm the presence of β2 subunits on nerves in these fibers, however staining for α4 subunits has not been completed. Type IV receptors are composed of α2β4/α4β4 subunits and while the staining results confirm the presence of α2 subunits, staining for the β4 subunits has not been completed. The pharmacologic data is consistent with type III receptors mediating nicotine induced relaxation in sling fibers. Type III receptors are composed of α3β4β2 subunits and the staining shown above confirms the co-localization of α3 and β2 subunits in nerves in sling fibers.

The present invention is not limited to the embodiments described and exemplified above, but is capable of variation and modification within the scope of the appended claims.

What is claimed:

1. A method for identifying modulators of gastroesophageal smooth muscle relaxation comprising:
inducing a fiber to contract,
contacting the fiber with an agent that induces the fiber to relax and a test compound, and
determining a modulation of the relaxation of the fiber in the presence of the test compound relative to the relaxation of the fiber in the absence of the test compound,
wherein the fiber is selected from the group consisting of: a clasp fiber isolated from the gastric smooth muscle of a mammal, a sling fiber isolated from the gastric smooth muscle of a mammal, a lower esophageal circular fiber isolated from the esophageal smooth muscle of a mammal, a mid esophageal circular fiber isolated from the esophageal smooth muscle of a mammal, and a mid esophageal longitudinal fiber isolated from the esophageal smooth muscle of a mammal.

2. The method of claim 1, wherein the agent is a nicotinic acetylcholine receptor agonist.

3. The method of claim 2, wherein the agonist is a hormone.

4. The method of claim 2, wherein the agonist is acetylcholine, carbachol, or nicotine.

5. The method of claim 1, wherein the fiber is contracted by contacting the fiber with muscarinic receptor agonist.

6. The method of claim 5, wherein the muscarinic receptor agonist is bethanechol.

7. The method of claim 1, wherein the mammal is a human cadaver.

8. The method of claim 1, wherein the fiber is contacted with the agent before the fiber is contacted with the test compound.

9. The method of claim 1, wherein the fiber is contacted with the agent and the test compound substantially at the same time.

10. The method of claim 1, wherein the fiber is contacted with the agent after the fiber is contacted with the test compound.

11. The method of claim 1, further comprising contacting the fiber with an agent that induces the fiber to relax and a negative control compound to provide a reference value for the level of modulation of the relaxation of the fiber induced by the test compound.

12. The method of claim 1, further comprising contacting the fiber with an agent that induces the fiber to relax and a positive control compound to provide a reference value for the level of modulation of the relaxation of the fiber induced by the test compound.

13. A method for identifying modulators of gastroesophageal smooth muscle relaxation comprising:
expressing at least one nicotinic acetylcholine receptor in a cell, wherein the receptor comprises one or more subunits, wherein each subunit is independently an alpha subunit, a beta subunit, a gamma subunit, a delta subunit, or an epsilon subunit,
contacting the receptor with a test compound, and
determining a modulation of the biological activity of the receptor in the presence of the test compound relative to the biological activity of the receptor in the absence of the test compound.

14. The method of claim 13, wherein the receptor comprises at least one alpha subunit and at least one beta subunit.

15. The method of claim 14, wherein the at least one alpha subunit comprises an alpha-2 subunit, an alpha-3 subunit, an alpha-4 subunit, an alpha-5 subunit, an alpha-7 subunit, an alpha-9 subunit, an alpha-10 subunit, or a combination thereof.

16. The method of claim 14, wherein the at least one beta subunit comprises a beta-2 subunit.

17. The method of claim 13, wherein the receptor comprises at least five subunits.

18. The method of claim 17, wherein the receptor comprises at least two alpha subunits, at least one beta subunit, at least one gamma subunit, and at least one delta subunit.

19. The method of claim 17, wherein the receptor comprises at least two alpha subunits, at least one beta subunit, at least one epsilon subunit, and at least one delta subunit.

20. The method of claim 13, adapted for high throughput screening.

21. The method of claim 13, further comprising contacting the receptor with a nicotinic acetylcholine receptor agonist.

22. The method of claim 13, wherein the cell is a eukaryotic cell.

23. The method of claim 22, wherein the eukaryotic cell is a yeast, mammalian, or insect cell.
